(19) United States
(12) Patent Application Publication
(10) Pub. No.: US 2024/0003911 A1
(43) Pub. Date: Jan. 4, 2024

(54) QUANTIFICATION OF SPERM CENTRIOLE QUALITY

(71) Applicant: The University of Toledo, Toledo, OH (US)

(72) Inventors: Tomer Avidor-Reiss, Toledo, OH (US); Emily Fishman, Toledo, OH (US); Katerina Turner, Toledo, OH (US)

(73) Assignee: The University of Toledo, Toledo, OH (US)

(21) Appl. No.: 18/038,053

(22) PCT Filed: Nov. 22, 2021
(86) PCT No.: PCT/US2021/060290

§ 371 Date: May 22, 2023

Related U.S. Application Data
(60) Provisional application No. 63/117,056, filed on Nov. 23, 2020.

(51) Int. Cl. G01N 33/68; G01N 33/58; G01N 21/64

(52) U.S. Cl. CPC G01N 33/6893; G01N 33/582; G01N 21/6428; G01N 2800/367; G01N 2021/6439

(57) ABSTRACT

Fluorescence-based ratiometric assessment of centrioles (FRAC), and methods of using the same and assays utilizing the same, are described. FRAC allows for the diagnosis of male infertility by analyzing centriole quality of sperm.

### RELATED APPLICATIONS

This application claims priority to U.S. Provisional Application No. 63/117,056 filed under 35 U.S.C. § 111(b) on Nov. 23, 2020, the disclosure of which is incorporated herein by reference in its entirety for all purposes.

### STATEMENT REGARDING FEDERALLY SPONSORED RESEARCH

This invention was made with no government support. The government has no rights in this invention.

### BACKGROUND

Sperm centrioles are essential for embryo fertilization and development in invertebrates and lower vertebrates. However, the precise role of human sperm centrioles in pre-fertilization and the essential role of human sperm centrioles in post-fertilization events is still debated. This controversy is due to two significant issues. First, most of the studies that indicate the centriole is essential after fertilization are of a small scale (Level III, Quality of Evidence). Available literature includes about a dozen case studies, case-control studies, and a few retrospective studies; no prospective studies have been reported. Most of these reports analyzed less than a handful of infertile patients—at most, ten patients. Also, the centriole was only directly examined in a few of these studies, and in many, centriolar defects are inferred from gross cell phenotypes. Second, sperm centrioles are absent in pre-fertilization mature mice spermatozoon and are not essential in post-fertilization in mice, and mice offspring can be generated from eggs fertilized by isolated sperm nuclei with no centrioles. However, the dispensability of sperm centrioles observed in mice does not appear to apply to humans, bovine, other domesticated animals, and many other mammals.

Assessment of sperm morphology is an important component of semen analysis and for the diagnosis of male factor infertility. Abnormal sperm morphology (teratospermia) is estimated to be present in 7-18% of infertile men. Yet, conventional sperm morphology assessment is inherently variable and subjective, and its predictive value for pregnancy success is controversial. However, the rates of pregnancy and live birth appear to be lower in some cases of teratospermia. Therefore, to better predict the potential for conception using teratospermic sperm, it may be useful to determine if the sperm has defects in its internal components, such as DNA, RNA, and centrioles. Consequently, there is a need for a robust method to analyze sperm components, including the centrioles.

About one-third of infertile couples have unexplained infertility and whether the man or women carry the deficiency leading to the infertility is unknown. Since centrioles are not currently diagnosed as part of infertility treatment, they may be an undiscovered couse.

Several assays to study human sperm centrioles are available. However, the associated technology is either laborious, not sufficiently specific to centrioles, or qualitative. The structure of sperm centrioles has been assessed by electron microscopy, an extremely laborious technique that is inadequate for large-scale studies and is inaccessible in most clinical settings. Sperm centriolar protein content has been assessed by western blotting, which measures total sperm protein and is not specific to the centriole; therefore, it cannot conclusively implicate the centriole in infertility. Sperm centriole function has been assessed by microinjection of human sperm into oocytes of bovine, rabbit, or hamster, followed by immunofluorescent staining for aster formation. These assays are useful for studying abnormal sperm centrioles in some infertility cases; however, since this method destroys a potentially viable embryo, it is not allowed in U.S. laboratories utilizing NIH funding (as per the Dickey-Wicker Amendment). Due to these limitations, there is a need for a high-throughput, specific, and quantitative method of assessing human sperm centrioles for defects.

### SUMMARY

Provided is a method for diagnosing a likelihood of infertility in a male subject, the method comprising staining a sperm sample from a male subject with at least one antibody configured to bind to a centriolar marker at locations comprising each of a proximal centriole, atypical distal centriole, and axoneme of the sperm (and optionally also in the striated column (SC)), wherein the antibody is configured to fluoresce upon binding to the centiolar marker; observing fluorescence from the stained sperm sample at each of the locations; comparing the observed fluorescence at each of the locations to a total fluorescence from the stained sperm sample to obtain a ratio at each of the locations for the centriolar marker; averaging the ratios by a number of samples to obtain a mean ratio at each of the locations; comparing each of the mean ratios to reference mean ratios at corresponding locations from a reference sperm sample, wherein the reference sperm sample has standard morphology or obtained from fertile donors; and diagnosing a likelihood of infertility in the male subject when any one of the mean ratios falls outside of two standard deviations from the reference mean ratio at the same location.

In certain embodiments, the centriolar marker comprises tubulin. In certain embodiments, the centriolar marker comprises POC1B. In certain embodiments, the centriolar marker comprises tubulin and POC1B. In certain embodiments, the centriolar marker comprises any of tubulin, acetylated tubulin, POC1B, POC5, CPAP, CEP63, CEP290, CETN1, CETN2, FAM161A, WDR90, CEP57, and CEP152. In certain embodiments, the centriolar marker comprises two or more markers selected from the group consisting of tubulin, acetylated tubulin, POC1B, POC5, CPAP, CEP63, CEP290, CETN1, CETN2, FAM161A, WDR90, CEP57, and CEP152. In certain embodiments, the centriolar marker comprises three or more markers selected from the group consisting of tubulin, acetylated tubulin, POC1B, POC5, CPAP, CEP63, CEP290, CETN1, CETN2, FAM161A, WDR90, CEP57, and CEP152.

In certain embodiments, the reference sperm sample comprises sperm from an infertile subject with standard morphology or fertile donors.

In certain embodiments, the antibody is labeled with a fluorescent tag. In certain embodiments, a secondary antibody is used to configure the antibody to fluoresce upon binding to the centriolar marker.

In certain embodiments, the male subject is a human. In certain embodiments, the male subject is a male ungulate species. In certain embodiments, the male subject is a male ruminant species. In certain embodiments, the male subject is a bull, ram, boar, dog, or horse. In certain embodiments, the male subject is a male mammalian food animal species. In certain embodiments, the male subject is a domesticated animal.

Further provided is a system comprising one or more antibodies configured to fluoresce upon binding to a centriolar marker selected from the group consisting of tubulin, acetylated tubulin, POC1B, POC5, CPAP, CEP63, CEP290, CETN1, CETN2, FAM161A, WDR90, CEP57, and CEP152; and a computer system configured to read fluorescence from a stained sperm sample, and process the fluorescence through an algorithm to calculate mean ratios of fluorescence at multiple locations within each sperm. In certain embodiments, the system further comprises a microscope.

Further provided is a method for selecting a sire for breeding, the method comprising analyzing sperm samples from a group of sires to determine centriole quality in the sperm samples, and selecting a sire for breeding from the group of sires based on the determined sperm centriole quality, wherein better sperm centriole quality indicates a likelihood of better fertility of the sire. In certain embodiments, the sperm centriole quality is determined through a fluorescence-based ratiometric analysis. In certain embodiments, the fluorescence-based ratiometric analysis determines centriole quality of each of a proximal centriole and an atypical distal centriole.

Further provided is an assay comprising one or more primary antibodies configured to bind to a centriolar marker selected from the group consisting of tubulin, acetylated tubulin, POC1B, POC5, CPAP, CEP63, CEP290, CETN1, CETN2, FAM161A, WDR90, CEP57, and CEP152; and one or more secondary antibodies configured to fluoresce upon binding to the one or more primary antibodies.

Further provided is a method for analyzing sperm, the method comprising separating sperm into a pellet and an interface using differential gradient centrifugation; separating the interface from media; washing the interface and the pellet with washing media; resuspending the interface and the pellet in PBS to produced suspended sperm; applying antibodies to the suspended sperm to stain the sperm; incubating the suspended sperm with secondary antibodies; imaging the suspended sperm with a microscope to analyze the staining of the suspended sperm; quantifying fluorescence from the suspended sperm to create mean ratios of the fluorescence at specific locations within the suspended sperm; and comparing the mean ratios to reference mean ratios of fluorescence from the specific locations within a reference sperm.

In certain embodiments, the antibodies comprise anti-POC1B, anti-tubulin, anti-acetylated anti-POC5, anti-CPAP, anti-CEP63, anti-CEP290, anti-CETN1, anti-CETN2, anti-FAM161A, and anti-WDR90. In certain embodiments, the secondary antibodies comprise donkey anti-rabbit DyLight 650, donkey anti-rabbit Alexa 488, donkey anti-Sheep Cy3, or donkey anti-Sheep Alexa 555.

Further provided is a method for diagnosing male infertility, the method comprising analyzing a sperm sample from a male subject to determine centriole quality in the sperm sample, wherein the centriole quality is determined with respect to each of a proximal centriole and an atypical distal centriole; comparing the determined centriole quality to centriole quality of a reference sperm sample from a fertile male reference subject; and diagnosing the male subject as infertile if the determined centriole quality is more than two standard deviations away from a mean from the reference sperm sample.

In certain embodiments, the analyzing is conducted through a fluorescence-based ratiometric analysis. In certain embodiments, the method further comprises treating the male subject for infertility.

Further provided is a kit comprising a first container housing a primary antibody configured to bind to a centriole selected from the group consisting of tubulin, acetylated tubulin, POC1B, POC5, CPAP, CEP63, CEP290, CETN1, CETN2, FAM161A, WDR90, CEP57, and CEP152; and a second container housing a secondary antibody configured to bind to the primary antibody. In certain embodiments, the kit further comprises a computer or a microscope.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIGS. 1A-1C: Quantitative imaging of sperm centriole markers. FIG. 1A shows an example phase and fluorescent image of a group of pellet spermatozoa. Such a picture ordinarily contains 10-30 spermatozoa. FIG. 1B shows a zoom-in on one sperm from FIG. 1A using HyVolution microscopy. FIG. 1C shows a zoom-in on the proximal centriole (PC), distal centriole (DC), and axoneme (Ax) of sperm from FIG. 1B labeled with anti-POC1B and anti-tubulin antibodies. Nucleus (N).

FIGS. 2A-2D: FRAC identifies sperm populations with suboptimal centrioles. For each panel, the mean and two standard deviations (SD) of the reference population are indicated to the right of each set of mean ratios. FIG. 2A shows mean ratios of the pellet sperm with standard morphology (the reference population) and pellet sperm from donors. The top of the graph indicates the skewness (Ske) and kurtosis (Kur) of the mean ratios distribution. FIG. 2B shows mean ratios of the interface sperm with standard morphology. FIG. 2C shows mean ratios of the pellet sperm with teratospermia (abnormal sperm morphology). FIG. 2D shows mean ratios of the interface sperm with teratospermia. Samples that were outside of 2 SD were identified as outliers (colored yellow and labeled). Outliers that were beyond 3 SD are colored red, and labeled. The semen analysis results of these outlier patients are identified in the upper left of each graph.

FIGS. 3A-3D: Individual sperm ratio distribution analysis provides additional insight into sperm centriole abnormalities. Histogram of the percent of sperm at different ratios of POC1B proximal centriole (PC), POC1B distal centriole (DC), and POC1B axoneme (Ax). Reference (blue) is the sperm of the 19 patients within the 22 patient reference population that have optimal centriole quality. FIG. 3A shows ratios of 44 pellet sperm from a patient, P28p (red), that does not have teratospermia but was found to have suboptimal POC1B ratios in the PC and DC. FIG. 3B shows ratios of 54 interface sperm from a patient, P28i (red), that does not have teratospermia but was found to have suboptimal POC1B ratios in the PC and DC. FIG. 3C shows ratios of 32 pellet sperm from a patient, P22p (red), that has teratospermia, and was found to have suboptimal POC1B ratios in the DC and Ax. FIG. 3D shows ratios of 77 interface sperm from a patient, P22i (red), that has teratospermia, and was found to have suboptimal POC1B ratios in the DC. The Histogram gates for the ratios on the X axis are 0-0.1, 0.1-0.2, 0.2-0.3, 0.3-0.4, 0.4-0.5, 0.5-0.6, 0.6-0.7, 0.7-0.8, 0.8-0.9, 0.9-1.

FIGS. 4A-4B: The DC's tubulin mean ratio correlates with that of DC's POC1B. FIG. 4A shows a graph depicting the correlation of optimal reference population. FIG. 4B shows a table summarizing the correlation P value (P), Pearson R value (R), and number of men (N).

FIGS. 5A-5C: Centriole staining abnormalities. FIG. 5A shows an example of standard staining from the pellet sperm of patient 19, who had a standard semen analysis, and whose centriole ratios fell within 2 SDs of the mean for all mean ratios. FIG. 5B shows an example of abnormal staining from the pellet sperm of patient 27, who had a severe oligoasthenoteratospermia phenotype such that the centrioles could not be quantified. FIG. 5C shows an example of abnormal staining from the pellet sperm of patient who had a asthenoteratospermia phenotype, and whose centriole ratios fell more than 3 SDs out of the mean for distal centriole POC1B. N, nucleus; PC, proximal centriole; DC, distal centriole; Ax, axoneme.

FIGS. 6A-6B: Characterization of the bull sperm centriole. FIG. 6A shows quantitative imaging of centriolar markers of a bull sperm. FIG. 6B, left panel, shows a low magnification image of a spermatozooan.

FIGS. 7A-7B: Sperm of sub-fertile sires have lower centriole quality. FIG. 7A shows mean±2SD distribution of the mean ratio for tubulin (Tub), Act-tubulin (ActTub), POC1B, and FAM161A at the PC (P), DC (D), and Ax (A) in the normal sperm populations of fertile sires. The y-axis represents the intensity ratio. FIG. 7B shows the mean ratio of abnormal sperm populations of fertile sires relative to the distribution of the normal sperm populations of fertile sires. In FIG. 7B, only outlier values are depicted.

FIGS. 8A-8B: Low quality centrioles associate with bull artificial insemination subfertility. FIG. 8A shows that an outlier comparison to the healthy fertile sperm (used as the reference population) found lower centriole quality in subfertile bulls' healthy sperm, subfertile bulls' unhealthy sperm, and fertile bulls' unhealthy sperm. Outlier bulls is the number of bulls with at least 1 outlier parameter. Double outlier bulls is the number of bulls with at least 2 outlier parameters. FIG. 8B depicts a graph showing the correlation between SCR deviation from the mean to the acetylated tubulin PC healthy sperm mean ratio. Bulls with outlier values are marked with their number of outlier parameters, the name of the outlier biomarker (Tb; Tubulin, AT; Ace Tubulin, PO; POC1B, FA; FAM161A), its location (PC, DC, or Ax), and arrows indicating if the number is higher (↑) or lower (↓) than the reference range. The number of arrows indicates the number of SDs away from the reference range's upper or lower bound (rounding up).

FIGS. 9A-9B: Dog and horse sperm have a proximal centriole (PC) and distal centriole (DC). The DC has two rods that are bigger than the PC lumen. FIGS. 9A-9B show low magnification images of sperm with dotted boxes marking the magnified neck depicted in the panels to the left. FIG. 9A shows a low magnification of dog sperm with a dotted box marking the magnified neck. Dog sperm centrioles were labeled by the microtubule protein tubulin and the centriole rod/lumen proteins FAM161A and POC1B. Tubulin also marks the axoneme of the flagellum. Three sperm cells are shown (i, ii, and iii). FIG. 8B shows a low magnification of horse sperm with a dotted box marking the magnified neck. Horse sperm centrioles were labeled by the centriole rod/lumen protein POC1B. Tubulin and FAM161A do not mark the axoneme and centrioles, likely because of issues with antibody specificity.

FIG. 10: Table showing a semen analysis of properties of fertile men and unexplained infertility patient populations. Fertile men had prior paternity with normal semen analysis.

FIGS. 11A-11F: FRAC identifies suboptimal centrioles in unexplained infertility. FIG. 11A shows a breakdown of 4 populations used. FIG. 11B shows mean rations of the fertile pellet sperm and their reference range. FIG. 11C shows mean ratios of the fertile interface sperm relative to the reference range. FIG. 11D shows mean ratios of the pellet sperm with unexplained dinfertility relative to the reference range. FIG. 11E shows mean ratios of the interface sperm with unexplained infertility relative to the reference range.

FIG. 12: FRAC identifies three levels of centriole quality in unexplained infertility men. FIG. 12 shows a table of mean ratios of healthy and unhealthy sperm from unexplained infertility patients. Blue font indicates a mean ratio that is <1 SD over the reference range. Red font indicates a mean ratio that is >1 SD over the reference range. Yellow highlights mark the “possible” level. Orange highlights mark the “likely” level.

FIG. 13: FRAC identifies suboptimal centrioles in multiple forms of infertility. FIG. 13 shows a graph showing the percent of outlier parameters and outlier men in healthy sperm samples of fertile men, unexplained couple infertility (Unexplained), infertile men with eumorphic sperm (Eumorphic), and infertile men with teratospermia sperm (Teratospermia). The fraction of outlier over total is indicated above each column. Significantly different samples based on Z-test are highlighted by a line with * (P<0.05), ** (P<0.01), and *** (P<0.001). Z-tests were calculated.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Centrioles are essential for cell function and embryo development. Irregular centrioles are implicated in multiple diseases such as cancer and infertility. However, the prevalence or extent of irregular centrioles in many diseases is unknown due to the lack of a convenient way to assess centriole quality. To overcome this roadblock, provided herein is a robust, location-based, ratiometric assay, referred to as fluorescence-based ratiometric assessment of centrioles (FRAC).

Sperm centrioles are essential for the formation and movement of the spermatozoon, and the sperm is the sole contributor of centrioles to the zygote. Therefore, centrioles are essential for human fertility. The human spermatozoon has two centrioles: Proximal Centriole (PC), which is found near the sperm head, and Distal Centriole (DC), which is further away from the sperm head and nucleates the axoneme. In the male germ cells that mature into spermatozoa, these centrioles undergo a unique “centriole remodeling”, which results in sperm centrioles having distinct, atypical characteristics. The spermatozoon PC has a canonical structure but somewhat atypical composition compared to the centrioles in other cells. The sperm DC has a dramatically atypical structure and composition compared to centrioles in other cell types, and it was only recently identified in human spermatozoon and other mammals.

The important role of the centrioles in sperm morphology and movement pre-fertilization is well demonstrated and widely accepted. The sperm centriole forms the flagellum (i.e., the sperm tail), and later it controls the shape of the tail beating. Defects in these functions can result in teratospermia, oligospermia, asthenospermia, or a combination of these phenotypes. Although some of the essential functions of sperm centrioles are known, the precise role of the canonical PC and atypical DC in the mature spermatozoon is still unclear. Furthermore, infertility stemming from a defective PC or DC may not be able to be circumvented by intracytoplasmic sperm injection (ICSI) with ejaculated or testicular sperm (e.g., testicular sperm extraction, TESE). Because of the impact on infertility treatments, there is a need for a more direct assessment of the role of human sperm centrioles, and FRAC may fill this need.

FRAC is a robust and convenient assay for centriole quality. In the examples herein, FRAC was utilized in the most extensive investigation of human sperm centrioles performed to date. It was found that infertile men with teratospermia have lower quality centrioles, although it is unclear what the causal relationship is between centriolar defects and morphology. It was also found that some men from infertile couples with unexplained infertility have lower quality centrioles, indicating these males are contributing to the couple infertility. These examples open the door for extensive characterization of the contribution of sperm centrioles to male infertility. Furthermore, the examples herein also show that subfertile bovine sires have lower quality sperm centrioles than fertile sires. Thus, FRAC can be used to identify better sires for breeding.

Advantageously, fluorescence microscopy provides a high-throughput technique to predict sperm function based on protein markers, and the centrioles can be specifically analyzed. Fluorescence microscopy has previously been used only to identify large defects that include protein mislocalization in small scale studies. However, in accordance with the present disclosure, FRAC can be used to assess the quality of both centrioles in sperm, the proximal centriole and the atypical distal centriole, as a poor quality of either centriole can cause infertility.

In some embodiments, FRAC may involve labeling a sperm sample with one or more antibodies configured to bind to centriolar markers. The centriolar markers are proteins that may include, but are not limited to, tubulin, tubulin with post-translational modifications such as tubulin, acetylated tubulin, POC1B, POC5, CPAP, CEP63, CEP290, CETN1, CETN2, FAM161A, WDR90, CEP57, CEP152, and combinations thereof. In some non-limiting examples, tubulin and POC1B are used as centriolar markers. Tubulin and POC1B are structural components of the centriole and axoneme. Tubulin is a heterodimer complex that polymerizes to form the centriole and axoneme wall. POC1B is an evolutionarily conserved centriole protein that forms a luminal scaffold structure in canonical centrioles and rod structures in the atypical DC. Members of the POC1 family of proteins are important for centriole stability. The relationship between the microtubules and POC1B rod structures in the DC is unknown, but it likely to have a scaffolding role.

The antibodies may be directly labeled, or secondary antibodies may be utilized so as to cause detectable fluorescence from the bound antibodies. After staining the sperm sample with the antibodies, the fluorescence intensity is observed at each of multiple locations within the sperm: the proximal centriole (PC), the atypical distal centriole (DC), and the axoneme (Ax). A ratio may be calculated at each location by dividing the fluorescence intensity at the location over the total fluorescence intensity from all the locations for each centriolar marker (i.e., [location/(PC+DC+Ax)] for each marker). The mean ratio is the average of a number (e.g., 50) of ratios per sample. The calculated mean ratios can then be compared to reference mean ratios from a reference sample, where the reference sample includes sperm having normal morphology or are from fertile donors. When any of calculated mean ratios at any of the locations in the sample is outside two standard deviations from the reference sample at the same location, then there is a likelihood of infertility.

Sperm centriole quality deteriorates at a rate of 1.2% per year in infertile men with normal sperm morphology. Most (˜86%) infertile men with normal sperm morphology have optimal sperm centrioles. However, most (˜78%) infertile men with teratozoospermia (abnormal sperm morphology) have sub-optimal sperm centrioles. FRAC may be used to diagnose male infertility based on sperm centriole disfunction.

The use of FRAC has numerous advantages. FRAC can be a diagnostic for centriole-based infertility, and is a robust and convenient assay for centriole quality. FRAC can be used in a routine clinical environment. FRAC allows infertility labs to adopt quantitative diagnosis of sperm quality to prevent unnecessary treatments and provide a more complete semen and sperm analysis. FRAC has a high throughput. The process can be streamlined for 100 or more samples per day. An automated algorithm can be implemented to analyze the images in a cost-effective, streamlined process.

Furthermore, FRAC may be utilized in the context of other animals, such as bulls. Reproduction is a major factor in farm profitability. Domestic animal reproduction in the United States is a major industry, with more than 9 million dairy cows being produced every year. FRAC can be used to help select sires for breeding based on sperm quality.

FRAC may be performed through a system that may include automatic slide staining functionality, automatic imaging, and automatic FRAC quantification. In some embodiments, a system may include antibodies for staining sperm samples, a computer, and a microscope, where the computer is configured to read stained sperm samples and calculate mean ratios using an algorithm.

The methods described herein may also be made available via a kit containing one or more key components. A non-limiting example of such a kit comprises a primary antibody in one container, and a secondary antibody in another container, where the primary antibody is configured to bind to a centriolar marker such as tubulin, acetylated tubulin, POC1B, POC5, CPAP, CEP63, CEP290, CETN1, CETN2, FAM161A, WDR90, CEP57, or CEP152, the secondary antibody is configured to bind to the primary antibody, and where the containers may or may not be present in a combined configuration. Many other kits are possible, such as kits further comprising a microscope or a computer. The kits typically further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

### Example I—FRAC Evaluation of Human Sperm Centrioles

Using the FRAC assay, a case series study was conducted with semen samples from 33 men, which were separated into higher-grade (pellet) and lower-grade (interphase) sperm fractions using differential gradient centrifugation. A narrow mean ratio range was found for the centriole markers POC1B and tubulin in the pellet of infertile men with morphologically standard sperm. FRAC identified 14% (3/22) of infertile men in this population have sperm with substandard centrioles. The interface sperm fractions of this population had 44% of samples with suboptimal sperm centrioles (4/9, p=0.06), indicating that lower-quality sperm also have low centriole quality. Fertile men had optimal centrioles in pellet sperm (N=2). In contrast, 79% (7/9) of the infertile men with substandard sperm morphology had suboptimal centrioles (p=0.0005). Altogether, these findings indicate that FRAC is a sensitive method for evaluating centriole quality, and that sperm with abnormal morphology have suboptimal centrioles.

Results

During the initial characterization of protein levels in the sperm centrioles using quantitative immunofluorescence (photon counting), it was found that immunofluorescence intensity values alone are not sufficiently sensitive to detect differences in staining across multiple studies. This insensitivity is largely due to the inherent variability of staining and imaging and appeared unrelated to the biological quality of the sperm. Therefore, the fluorescence-based ratio assessment of centrioles (FRAC) assay that compares the ratios of immunofluorescence intensity was developed. In this assay the intensity at different locations is compared to the total intensity of those locations (location/PC+DC+Ax for different markers (tubulin or POC1B)). The use of ratios is advantageous because it has the ability to isolate sperm-specific variables from experimental variation (i.e., lot-to-lot variation in antibodies, reagent potency, human error, and other variables that are difficult to control). The mean of these ratios was calculated from multiple sperm in a population to characterize the typical variability present in a sperm population of each man. The six individual mean ratios will be referred to by the location and protein (e.g., “POC1B PC” refers to POC1B PC/(POC1B PC+POC1B DC+POC1B Ax)).

FRAC was developed by immunostaining the centrioles and axoneme using two markers: the centriole specific marker POC1B labels both typical proximal centriole and atypical distal centriole, and the microtubule marker tubulin, which labels the PC, DC, and axoneme (Ax). As such, whether POC1B and tubulin can identify outlier values that may, in turn, identify sperm centriole associated disorders (SCARD) was evaluated.

Population Definitions and Assumptions

For the experiments, the sperm of 31 patients and two donors across all three studies was analyzed using mean ratios to minimize technical differences between studies. These 31 patients were all undergoing infertility treatments but had varying diagnoses; their sperm phenotypes were classified into men with standard morphology (population n=22) or those with teratospermia (population; n=9). Every sample was processed using gradient centrifugation, which resulted in two fractions. A pellet of dense sperm, which is generally regarded as of better quality, is used for intrauterine insemination, IUI. An accumulation of less dense sperm at the interface of the gradient phases, which has lower quality sperm, is the second fraction. The pellet sperm of every sample was collected (n=31), and the interface from studies 2 and 3 was also collected (n=19). This centrifugation produced four populations of sperm, each with distinct quality levels: a) the optimal pellet sperm from men without teratospermia (n=22) (the reference population); b) the sub-optimal interface sperm from men without teratospermia (n=9); c) the pellet sperm from men with teratospermia (n=8); and d) and interface sperm from the men with teratospermia (n=8). These four groups allowed for the characterization of the differences in sperm centriole quality between men with and without teratospermia, and the examination of the effectiveness of differential centrifugation in isolating sperm with quality centrioles.

To overcome the limited availability of fertile donor sperm, pellet sperm of infertile men with standard morphology was utilized. It was assumed that sperm centriole defects in this specific population of infertile men would be minimal. After calculating ranges from the sperm population of the pellet from men with standard morphology, the two donors were compared to this population to assess whether the values were similar to the pellet sperm from men with standard morphology. Since sperm population is highly heterogeneous, it was assumed that even the most fertile donors would have some sperm with poor centrioles, and the most infertile patients would have some sperm with good centrioles. While infertile patients are not an ideal population for gaining insight into the healthy, fertile population, data from infertile men can be used to compare distinct populations of infertile men, namely those with standard sperm morphology and those with abnormal sperm morphology. A similar approach was used to determine the differences between primary and secondary infertility in men with varicocele. The pellet sperm from the infertile men without teratospermia will be referred to as the “reference population”.

Most Infertile Men with Standard Morphology Sperm have Optimal Centriolar Mean Ratios

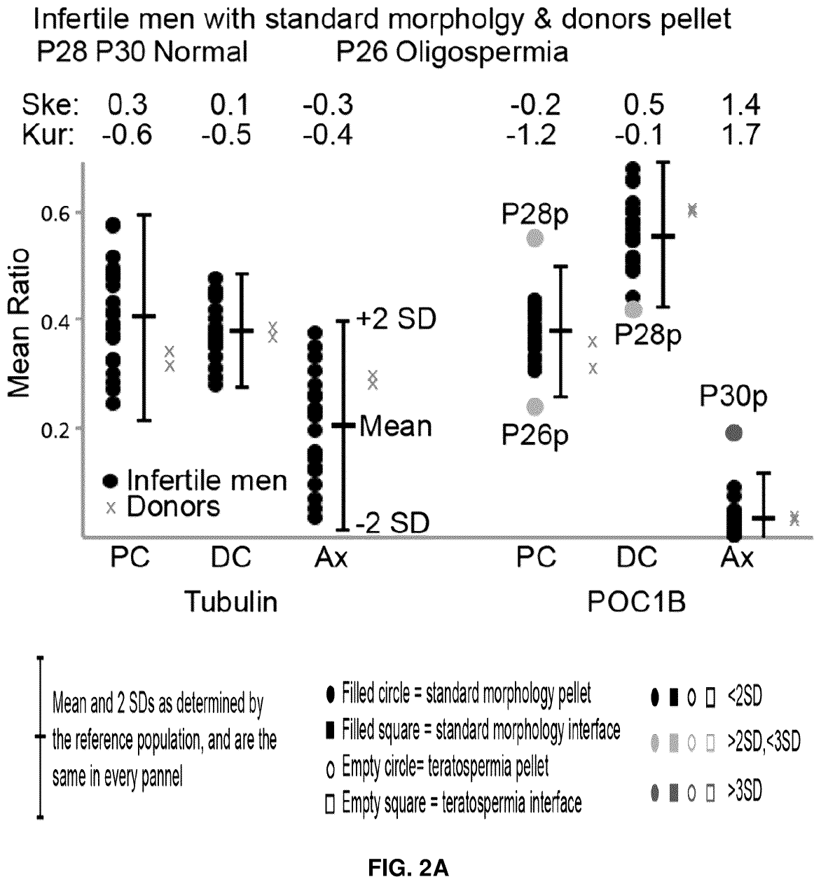

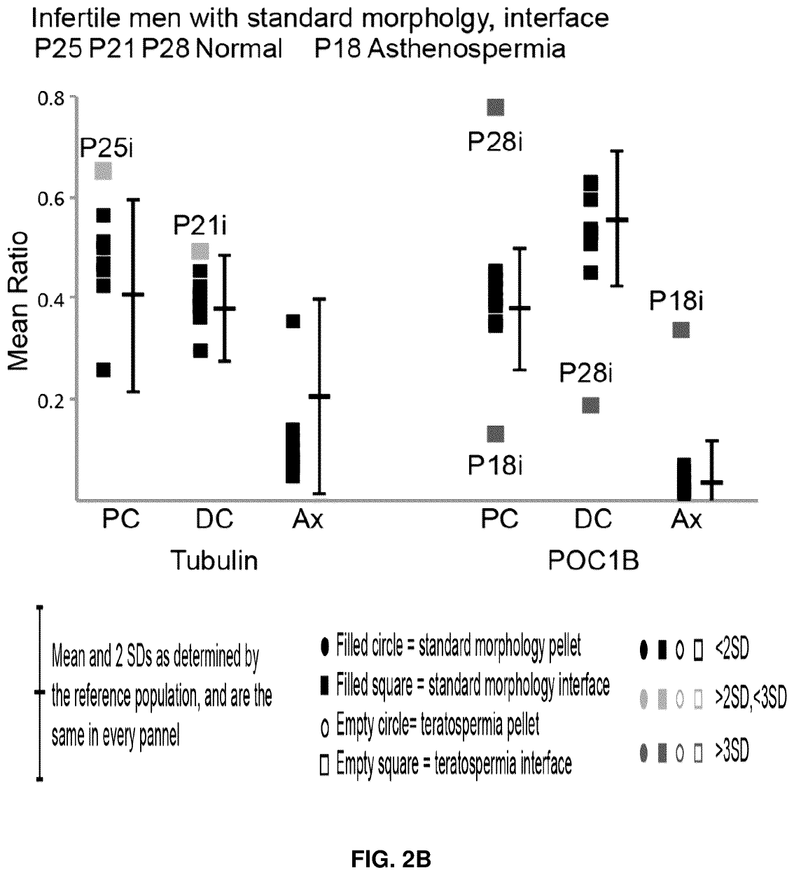

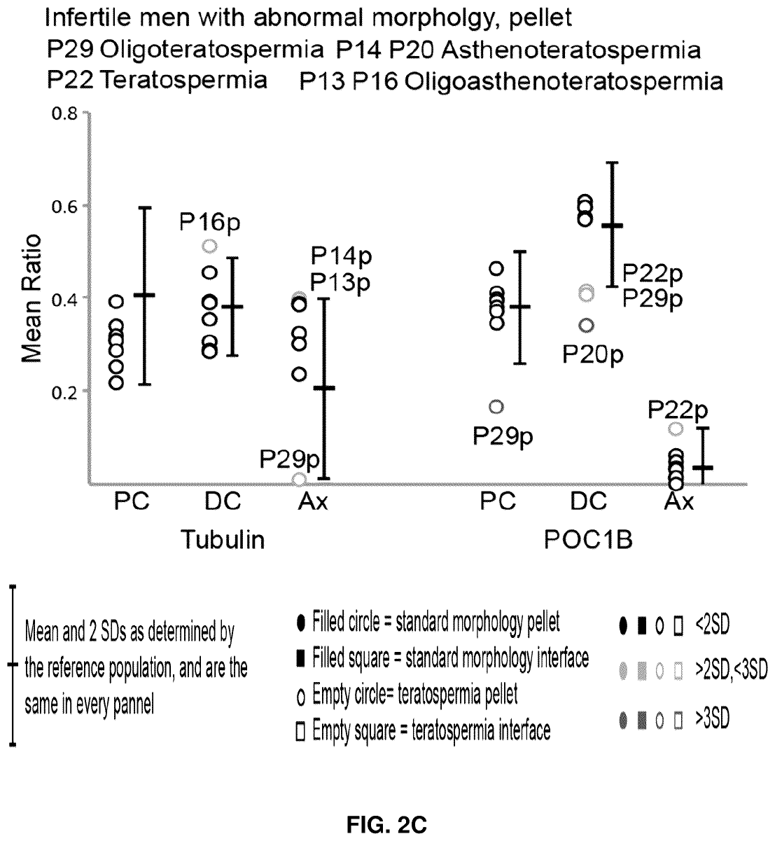

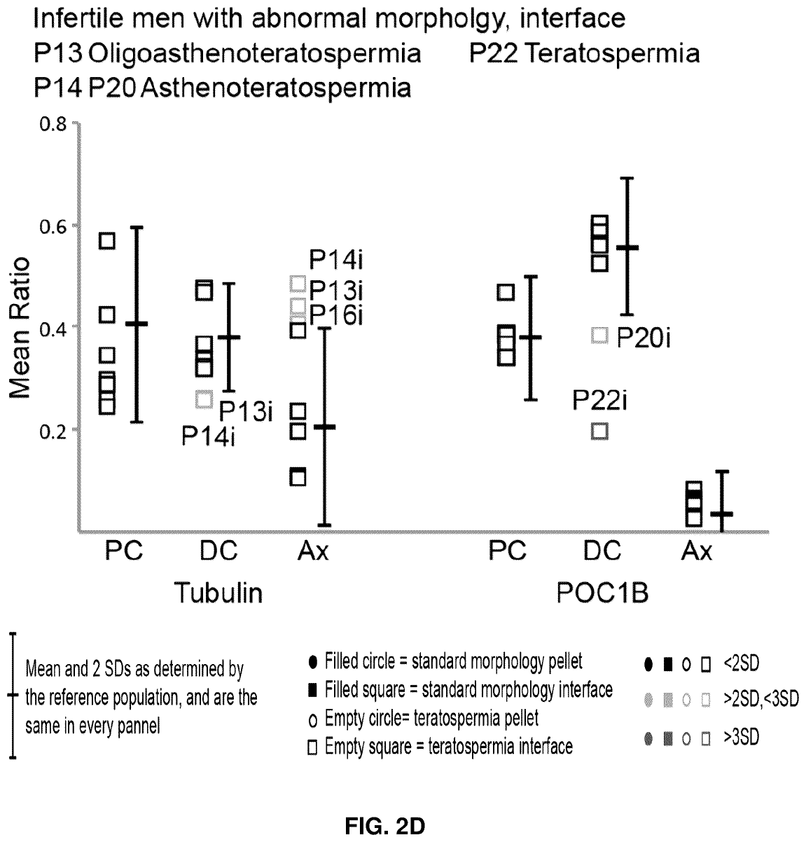

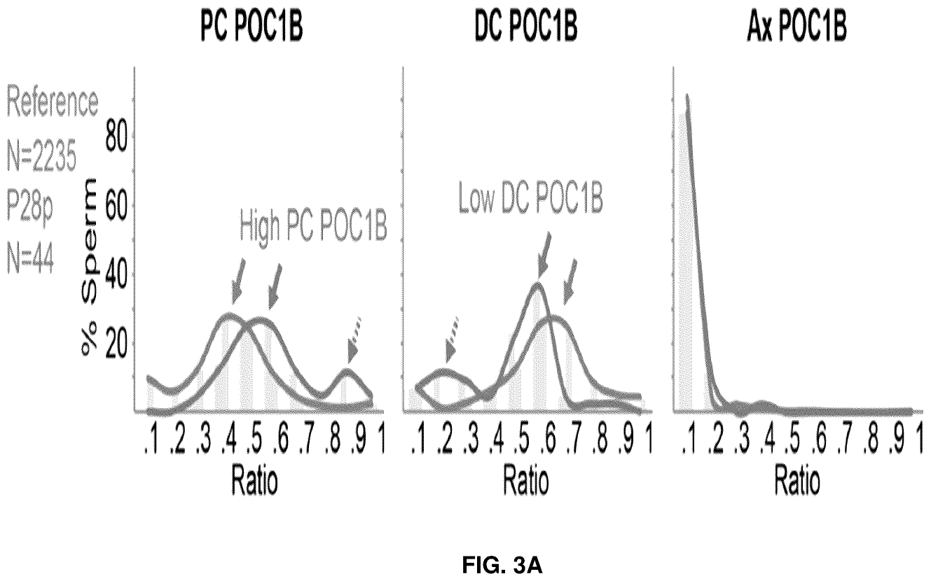

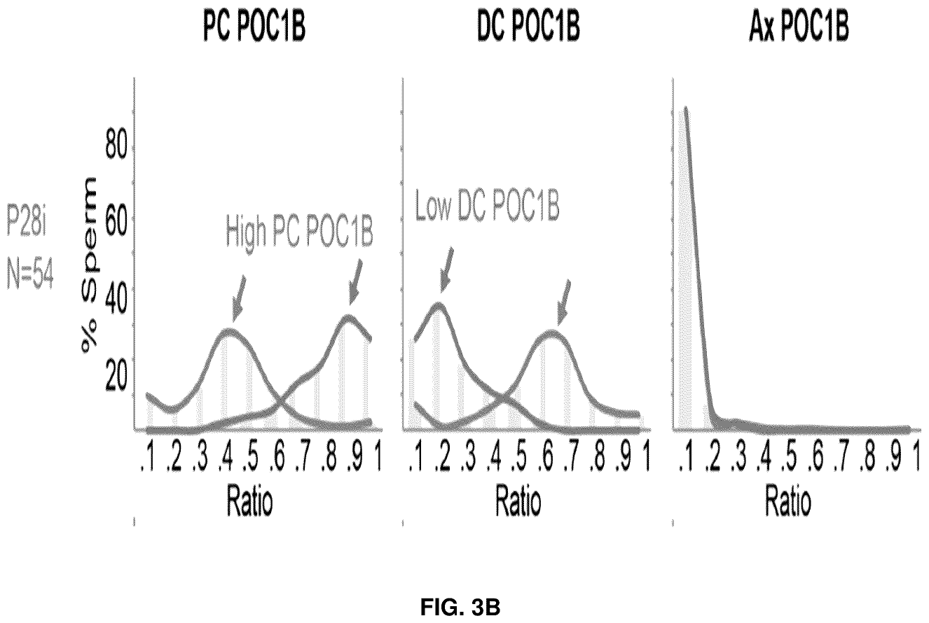

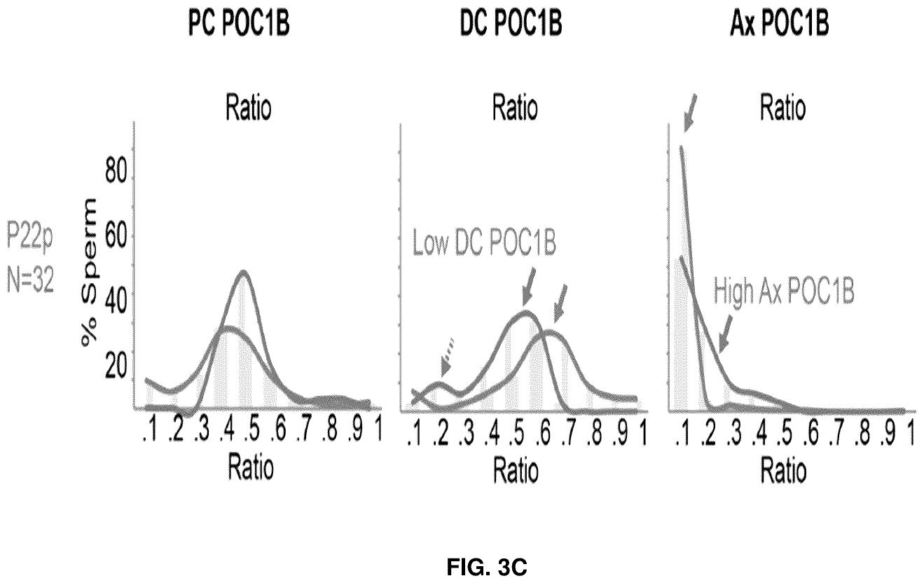

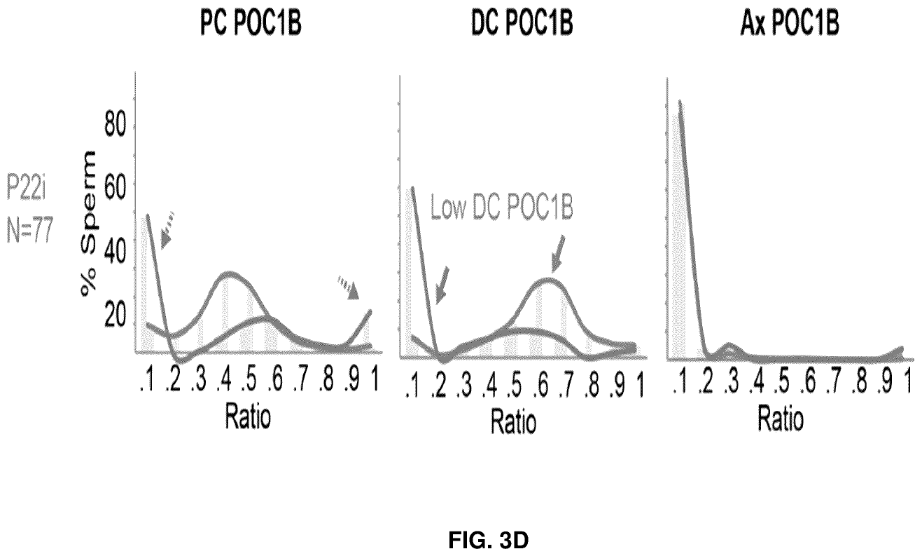

The variation of the mean ratio within the reference population was analyzed to identify outlier values. Two SDs away from the mean defines the samples as having a 95% probability of belonging to a common population, and this calculation is often used to determine a reference population. Therefore, the typical range of values present in the reference population samples was determined by calculating the mean of the ratios in the reference population±two SDs, which is referred to as the reference range (FIG. 2A). Samples outside the reference range were considered outliers and thus deemed to have sub-optimal centrioles. A sample from the reference population was considered to have optimal sperm centrioles if all six sperm mean ratios were within the reference range. Nineteen of the twenty-two samples (86%) from the reference population had all six mean ratios fall within the reference range and were considered to have optimal sperm centrioles and were later used in the distribution analysis and defined as the optimal reference population (FIGS. 3A-3D).

A sample with an outlier mean ratio in any of the six mean ratio locations/markers was considered as having sub-optimal sperm centrioles. Therefore, it was concluded that the remaining three samples from the reference population had sub-optimal sperm centrioles. Two of them, P26p and P28p, had mean ratios, between two and three SDs from the mean. The third patient, P30p, had a mean ratio of more than three SDs from the mean. P26p, who has a semen phenotype of oligozoospermia, had low PC POC1B; P28p, who has a semen phenotype of normospermia, had high PC POC1B and low DC POC1B. The third patient, P30p, who has a semen phenotype of normospermia, had high Ax POC1B. This result indicates that 14% (3/22) of the reference population samples have suboptimal centrioles. All six mean ratios of the two donor men analyzed fell within 2 SDs of the mean (FIG. 2A). This distribution indicates that the optimal reference population resembles fertile men (FIG. 2A).

POC1B has a Narrower Mean Ratio Range than Tubulin in the Optimal Reference Population

The distribution of the optimal reference population (FIG. 2A) was analyzed. It was found that the skewness (the degree of asymmetry of a distribution around its mean) is below is +/−1 for tubulin of PC, DC, and Ax, as well as for POC1B of PC and DC. Axoneme POC1B skewness was slightly larger, likely because POC1B usually is not found in the axoneme, and the distribution is one-sided. The kurtosis (sharpness of the peak around the mean) is below is +/−3 for tubulin of PC, DC, and Ax as well as for POC1B of DC and AC. Together, this indicates that the optimal reference population has an almost normal distribution.

The quality of a marker is defined by several factors, including narrowness of the range, sensitivity, and specificity. Currently, there is no way to determine the sensitivity and specificity of centriole quality, as these factors cannot be evaluated without prospective studies. To evaluate the narrowness of the range, the mean ratio ranges for the optimal reference population were determined for tubulin and POC1B at each location. The mean ratio ranges were determined by the difference between the highest mean ratio and the lowest mean ratio calculated as a percentage (i.e., in the PC, mean ratios for tubulin range from 0.25 to 0.57, the calculation would then be (0.57−0.25)*100=32%). The mean ratio ranges for Tubulin were 32% in the PC, 20% in the DC, and 36% in the Ax. The mean ratio ranges for POC1B were 13% in the PC, 19% in the DC, and 8% in the Ax. These values were used to calculate the overall mean ratio range for tubulin, which was 29%, and the overall mean ratio range for POC1B, which was 13%. This overall difference is statistically significant (P=0.05), indicating that POC1B is a better marker than tubulin based on this criterion.

The Ratio of DC's Tubulin Correlates with that of DC's POC1B

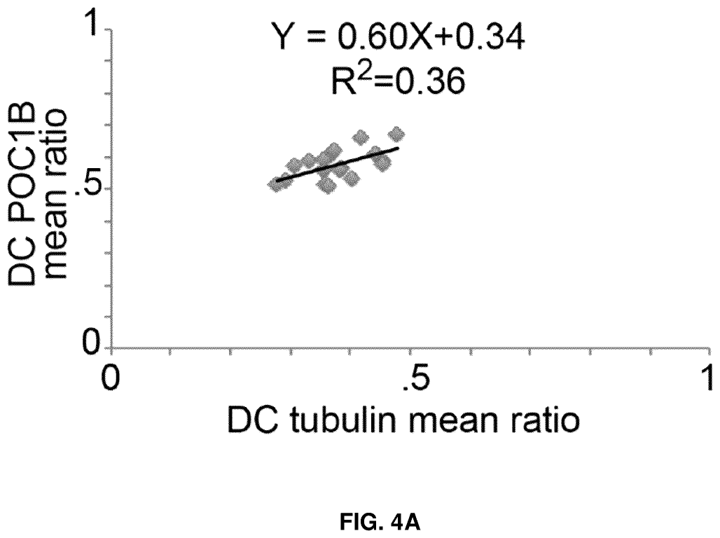

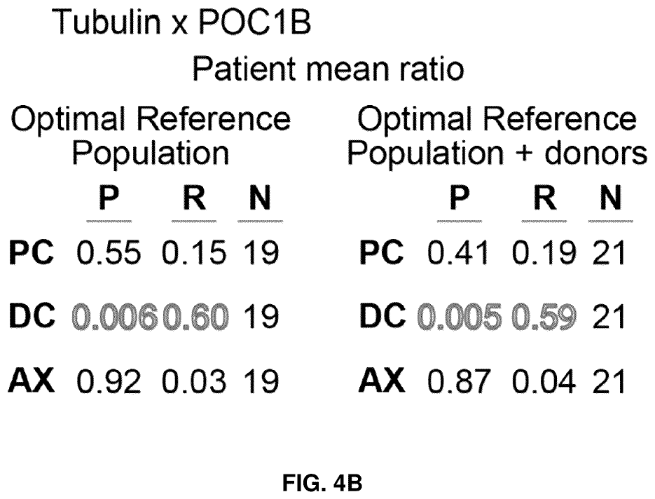

In canonical centrioles, POC1B forms a scaffold structure that is attached to the microtubules and controls centriole length. Therefore, whether the mean ratios of POC1B and tubulin correlate in the pellet of the optimal reference population with normal sperm morphology was analyzed. It was found that, in the DC, POC1B and tubulin have a significant and modest positive correlation with each other (P=0.006, R=0.60) (FIG. 4A). No significant correlation was observed between POC1B and tubulin in the PC or Ax (FIG. 4B). This data indicates that −30% of the DC tubulin ratio can be predicted by DC POC1B, and vice versa. A similar significant, positive correlation was obtained between POC1B and tubulin when the two donor samples were included (P=0.005, R=0.59) (FIG. 4B).

Interface Sperm of Infertile Men with Standard Morphology have Sub-Optimal Centrioles

As sperm cells differentiate, they lose cytoplasm and become denser. This difference is used to select better sperm as the pellet in differential centrifugation. During the same differentiation process, centrioles undergo remodeling. Therefore, it is believed that the less dense, and thus less mature, sperm found in the interface have reduced centriole quality. As such, the objective of this analysis was to test if POC1B and tubulin can identify differences between the centrioles in the interface and the pellet sperm populations.

The centrioles of the interface sperm from the nine infertile men with standard morphology were analyzed (FIG. 2B and Table 1). It was found that four samples from these men had outlier mean ratios of centriole staining (44% of this population). Two of them, P21i and P25i, had mean ratios between the two and three SDs away from the mean. P21i, who has a semen phenotype of normospermia, had a high level of DC tubulin. P25i, who has a semen phenotype of normospermia, had a high level of PC tubulin. The other two outlier samples, P18i and P28i, had mean ratios outside three SDs from the mean. P18i, who has a semen phenotype of asthenospermia, had low PC POC1B and high Ax POC1B. P28i, who has a semen phenotype of normospermia, had high PC POC1B and low DC POC1B. This patient (P28) seemed to have severely sub-optimal centrioles in both the interface and pellet; he had four outlier values between both sperm populations (pellet and interface), two of which were outside the 3-SD of the mean. These results indicate that the interface sperm of infertile men with standard morphology tend to contain more sperm with reduced quality centrioles than the sperm in the pellet fraction (5/10 versus 3/22, P=0.06) (Table 3).

TABLE 1
Summary table comparing the various populations of men examined in the study.
p-Value as determined by Z-test. SD, standard deviations; N, number; P, p-value; and NA, not available.

| | | | | Donor |
|---|---|---|---|---|
| | Infertile Standard | Infertile Abnormal | | Standard |
| | Morphology N | Morphology N | p-value: Infertile | Morphology |
| | (patients) (% of | (patients) (% of | Standard/Infertile | N (% of |
| | population) | population) | Abnormal | poplulation) |
| Pellet | N = 22 | N = 9 | | N = 2 |
| 2SD | 19 (86%) | 2 (22%) | 0.0005 | 2 (100%) |
| >2SD | 3 (14%) | 7 (78%) | 0.0005 | 0 |
| >2SD, <3SD | 2 (P26, P28) (9%) | 4 (P13, P16, P22, P14) (44%) | 0.0238 | 0 |
| >3SD or no staining | 1 (P30) (5%) | 3 (P20, P27, P29) (33%) | 0.03 | 0 |
| Interface | N = 9 | N = 9 | | NA |
| 2SD | 5 (56%) | 3 (33%) | 0.3421 | NA |
| >2SD | 4 (44%) | 6 (66%) | 0.3421 | NA |
| >2SD, <3SD | 2 (P21, P25) (22%) | 4 (P13, P14, P16, P20) (44%) | 0.31732 | NA |
| >3SD or no staining | 2 (P18, P28) (22%) | 2 (P22, P27) (22%) | 1 | NA |
| p-value: | | | | |
| pellet/interface | | | | |
| 2SD | 0.06288 | 0.59612 | | |
| >2SD | 0.06288 | 0.59612 | | |
| >2SD, <3SD | 0.32218 | 1 | | |
| >3SD or no staining | 0.13104 | 0.59612 | | |
| Total | N = 9 | N = 9 | | NA |
| (pellet + interface) | | | | |
| 2SD | 3 (33%) | 2 (22%) | 0.81 | NA |
| >2SD | 6 (67%) | 7 (78%) | 0.5961 | NA |
| >2SD, <3SD | 3 (P21, P25, P26) (33%) | 3 (P13, P14, P16) (33%) | 1 | NA |
| >3SD or no staining | 3 (P218, P28, P30) (33%) | 4 (p20, P22, P27, P29) (44%) | 0.3421 | NA |

78% of Infertile Men with Teratospermia have Reduced Centriole Quality

Most studies indicating human sperm centrioles have a role post-fertilization are from IVF studies with teratospermia sperm. Since these studies show teratospermia and centriole-associated embryonic failures, it is believed that a significant portion of infertile men with morphologically abnormal sperm have suboptimal sperm centrioles. As such, the objective of this analysis was to test if POC1B and tubulin can identify centriolar differences between sperm populations with and without morphological defects.

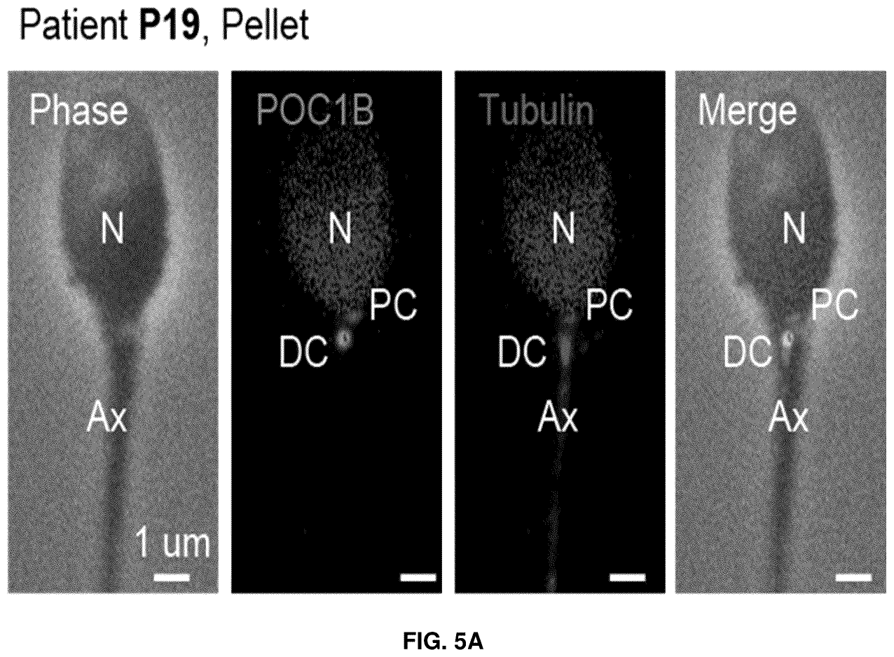

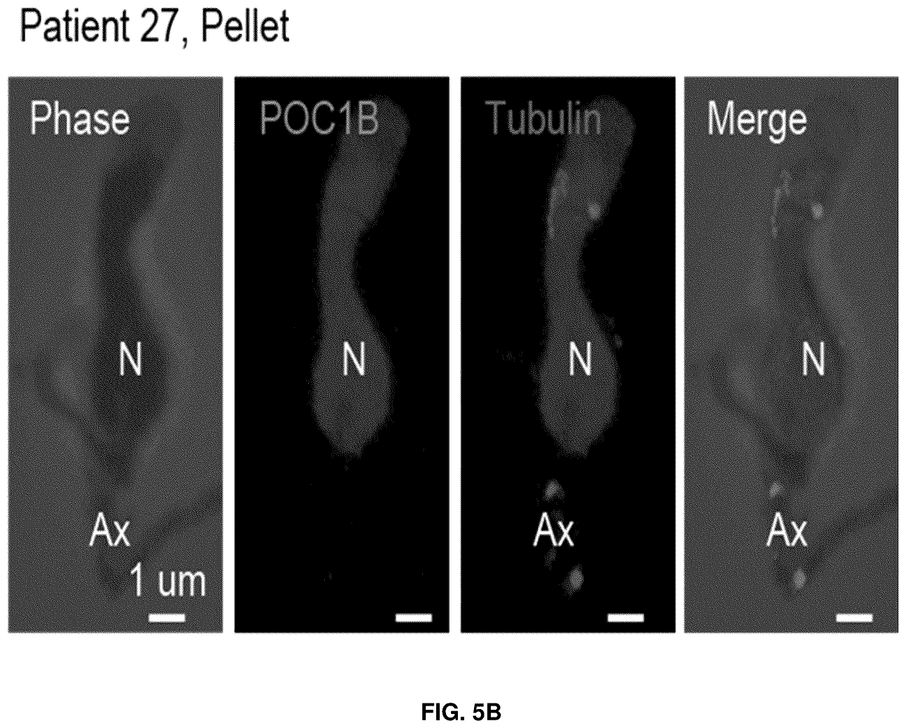

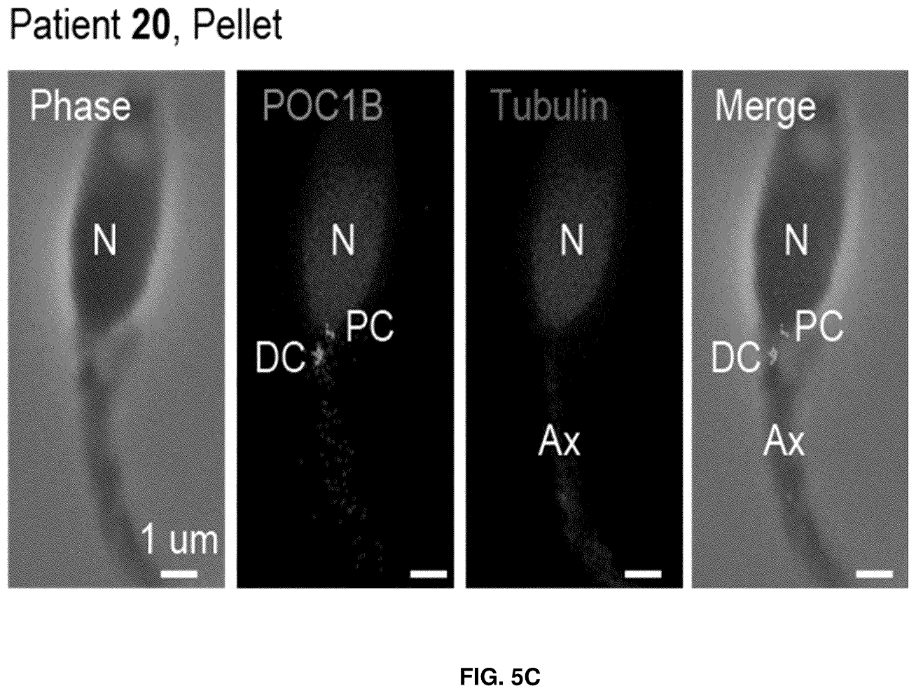

The staining of the pellet sperm from the nine patients with teratospermia (FIGS. 2C-2D, FIG. 5) was analyzed. For eight of the nine patients, the sperm centrioles using the antibodies were detected, and the mean ratio of their pellet sperm was analyzed (FIG. 2C). For one patient, P27, the PC and DC were not able to be detected as discrete entities using either POC1B or tubulin staining in the interface and pellet populations. Consequently, the mean ratios were not able to be analyzed (FIG. 5B). Therefore, it was concluded that the patient's sperm had a major centriole defect. Interestingly, this patient was one of three patients with the most defective sperm phenotypes, oligoasthenoteratospermia (OAT), in the samples studied as determined by semen analysis.

For the remaining eight of the nine teratospermia patients, it was found that six had outlier mean ratios of centriole markers (FIG. 2C). Four of these patients, P13p, P14p, P16p, and P22p, had mean ratios between the two and three SDs from the mean of the reference population. P13p, who has a semen phenotype of oligoasthenoteratospermia (OAT), had high Ax tubulin. P14p, who has a semen phenotype of asthenoteratospermia, had high Ax tubulin. P16p, who has a semen phenotype of oligoasthenoteratospermia (OAT), had high DC tubulin. P22p, who has a semen phenotype of teratospermia, had low DC POC1B and high Ax POC1B. Two of the patients, P20p and P29p, had mean ratios outside three SDs from the mean. P20p, who has a semen phenotype of asthenoteratospermia, had low DC POC1B. P29p, who has a semen phenotype of oligoteratospermia, had low PC and DC POC1B.

These results indicate that the patients with teratospermia have more sub-optimal centrioles than patients without teratospermia (7/9, 78%, versus 3/22, 14%, P=0.0005) (Table 2). Samples with mean ratios outside of 3 SDs or in samples where the centrioles were not able to be detected, P20p, P27p, and P29p, were designated as samples with “severe centriole defects.” It also indicates that there is a statistically significant difference in the number of severe centriolar defects (3/9 versus 1/22, P=0.03) (Table 3). This finding indicates that sperm with morphological defects are more likely to have lower quality centrioles.

Interphase and Pellet Sperm of Men with Teratospermia have Similar Sperm Centriole Quality

It was found that differential gradient centrifugation of sperm with standard sperm morphology results in a pellet enriched for better quality sperm centrioles. Whether differential gradient centrifugation has a similar effect on sperm from patients with teratospermia was evaluated. Interface sperm from men with teratospermia was analyzed (FIG. 2D). It was found that six of the nine interface sperm samples from men with teratospermia had mean ratios outside 2 SDs from the mean of the reference population. P13i, P14i, P16i, and P20i had mean ratios between the two and three SDs from the mean of the reference population. P13p, who has a semen phenotype of oligoasthenoteratospermia (OAT), had low DC tubulin and high Ax tubulin. P14p, who has a semen phenotype of asthenoteratospermia, had low DC tubulin and high Ax tubulin. P16p, who has a semen phenotype of oligoasthenoteratospermia (OAT), had high Ax tubulin. P20i, who has a semen phenotype of asthenoteratospermia, had low DC POC1B. One patient, P22i, had a mean ratio of more than three SDs away from the mean of the reference population. P22i, who has a semen phenotype of teratospermia, had a low DC POC1B. Again, the PC and DC were not able to be detected as discrete entities in patient P27, who has a semen phenotype of Oligoasthenoteratospermia, using POC1B or tubulin staining. This result indicates the infertile men with teratospermia have a similar incidence of sub-optimal centrioles in both interface and pellet fraction (7/9 versus 6/9 p is 1) (Table 1). This observation indicates that sperm selection by differential centrifugation is insufficient for increasing the odds of fertilization in cases of suboptimal centrioles, thus alternative selection techniques should be investigated.

Ratio Distribution Provides More Insight on Sperm Centriole Quality

A patient with optimal centrioles should have all its mean ratios within the reference ranges. However, the individual sperm of that patient may fall outside the reference ranges because sperm populations are heterogeneous. The inverse of this is also true: a patient with sub-optimal centrioles may also have some sperm with optimal centriole ratios. Therefore, to gain insight into the heterogeneity of the sperm samples, the POC1B ratio distribution of representative samples was analyzed. First, a reference distribution was determined. For that, a “master” distribution of all ratios from all the pellet sperm from the 19 men in the reference population that fell within two SDs (marked with blue in FIG. 3) was built, referred to as the reference distribution. In this reference distribution, POC1B ratios peak at a ratio of 0.3-0.4 for the PC, 0.6-0.7 for the DC, and 0-0.1 for the Ax. The POC1B PC and DC mean ratios had an approximately normal distribution, as the skewness and kurtosis were less than +/−1.0. Then, it was compared to the staining ratio distributions of P28p, P28i, P22p, and P22i.

Next, the pellet sperm sample of a patient with standard sperm morphology but with sub-optimal centrioles was analyzed. P28p has two outlier mean ratios in the pellet fraction: high PC POC1B and low DC POC1B (>2SD, <3SD). In P28p, the PC POC1B ratio peaks at 0.5-0.6 (red arrow in FIG. 3A, left panel), a pronounced shift to a higher ratio than the reference ratio distribution (blue arrow in FIG. 3A). Also, there is a smaller peak at a ratio 0.9, which is not present in the reference ratio distribution (dashed red arrow, FIG. 3A, left panel). Interestingly, the peak of DC POC1B ratio mirrors the PC POC1B shift; it was shifted to a lower range of 0.5-0.6 and had an additional peak from 0.1-0.3 (red arrows, FIG. 3A, middle panel). These shifts raise the possibility that the increased PC POC1B is at the expense of DC POC1B in P28p. Because these values are ratios, this reciprocal shift may be due to an increase in absolute POC1B staining in the PC or a reduction in absolute POC1B staining in the DC. The peak of the Ax POC1B ratio was not shifted (FIG. 3A, right panel).

P28 has the same two outlier mean ratios in the interface fractions, high PC POC1B and low DC POC1B, but it is more severe (>3SD) than the pellet fraction. In the interphase, the ratio peaks were shifted even further from the reference ratio distribution than the pellet sperm. The ratios for PC POC1B in P28i peaked between 0.9-1.0, which is a more exaggerated shift than the pellet population (FIG. 3B, left panel). DC POC1B ratios continue this trend and are shifted to lower than their pellet counterpart to 0-0.2 (FIG. 3B, middle panel). Therefore, this analysis indicates that the interface contains more sperm with sub-optimal centrioles in P28. The peak of the Ax POC1B ratio was not shifted, similar to that in the pellet (FIG. 3B, right panel).

Next, P22, who has a sperm phenotype of teratospermia, was analyzed. P22p has two mean ratio outliers in the pellet fraction: low DC POC1B and high Ax POC1B (>2SD, <3SD). In P22p, the DC POC1B ratio peaks at 0.5-0.6 (red arrow in FIG. 3C, middle panel), a shift to a lower ratio than the reference ratio distribution (blue arrow in FIG. 3C, middle panel). Additionally, there is a smaller peak at a ratio of 0.2, which is not present in the reference ratio distribution (dashed red arrow, FIG. 3C, middle panel). The peak of P22p's Ax POC1B ratio was shifted to a higher range of 0.2-0.3 than the reference ratio distribution at 0.1 (red arrow in FIG. 3B, right panel). These shifts indicate that the decreased DC POC1B is because of POC1B leaking from the DC into the Ax in P22p.

Surprisingly, in the interphase fraction, P22 was only identified to have one outlier mean ratio, low DC POC1B, but it is more severe (>3SD) than the pellet fraction. The ratio peak was further shifted from the reference ratio distribution in the interphase sperm (peaked at 0.1) than in the pellet sperm (red arrow in FIG. 3D, middle panel). Unexpectedly, even though the mean PC POC1B of P22i fell within the ratio range, the ratio distribution analysis shows very few sperm fall into the reference ratio distribution. Instead, there were two opposing peaks at 0-0.1 and 0.9-1 (the peak for the reference ratio distribution is 0.3-0.5). Therefore, the ratio distribution analysis finds that P22i contains sub-optimal centrioles that are missed by the mean ratio analysis.

Discussion

There is a need to develop an assay that can assess the sperm centriole quality in a clinical setting to reveal the contribution of sperm centriole to infertility. Here, this was performed by developing an assay that allows for the robust assessment of the quality of spermatozoa centrioles.

FRAC is based on immunofluorescence using sperm centriole markers. In this example, two markers were tested: tubulin and POC1B. However, this protocol can be extended to include markers of other protein components of the PC or DC (e.g., CETN1, POC5, and CEP135) as well as specific posttranslational modification (e.g., acetylated tubulin). Both abnormally low or high levels of any protein may indicate reduced centriole quality, as deviation in either direction was associated with sperm dysfunction.

Tubulin and POC1B are components of distinct structures. Tubulin is a structural protein of the microtubule; POC1B is a structural protein of the centriole lumen. Therefore, they report on distinct properties of the sperm centrioles. It was demonstrated that an immunofluorescent, localization-based, ratiometric, quantitative assay, such as FRAC, can give reasonably low variability between experiments that enabled the identification of outlier samples when comparing distinct sperm populations. This may be used to provide a comprehensive sperm quality report.

Here, infertile men were used, but FRAC can be used in fertile men. The reference range obtained from infertile men with standard morphology would resemble those obtained from fertile men, since sperm centriole defects are likely to affect only a small portion of infertility cases and the sperm from the two donors falls within the reference range, thus reinforcing this conclusion.

FRAC is faster than other techniques currently used to assess sperm centrioles. With automation and streamlining, it may be used routinely in a clinical setting. One way is to use automation of slide immunostaining. Alternatively, a higher prevalence of automation is possible with image-based flow cytometry. Automation of quantification is possible by writing an algorithm that scans the major features of the sperm, identifies the centrioles, reads the fluorescence intensity, and outputs a mean ratio.

One possible question regarding FRAC is whether it may not identify sperm that have an overall reduction in centriole staining since it is based on the relative amount of protein levels. However, this is unlikely as the two centrioles and the axoneme are distinct from each other, which indicates that the protein level in each structure is governed by different interactions with distinct affinities and avidities. Therefore, a significant reduction in protein expression would result in an uneven reduction in the various structures.

FRAC is based on image quantification, localization, and ratio calculation, and allows robust assessment of the spermatozoa centrioles. Measurement of the mean ratio of centriole and axoneme staining in the washed ejaculated spermatozoa may distinguish men with sub-optimal centrioles and teratospermia from men with just teratospermia. The mean ratio of spermatozoa in the ejaculate may be used to stratify whether infertile couples are recommended to use sperm donation rather than ICSI; additionally, women with lower ovarian reserve may store their eggs until the centriole quality of their male partner increases. FRAC is faster than other techniques currently used to assess sperm centrioles and therefore can become a useful tool in the clinical evaluation of semen quality. Applying the findings of this example to the sperm of infertile men, in general, indicates that a considerable fraction of infertile men have lower centriole quality.

Materials and Methods

This example was performed in three consecutive studies over three years (November 2016 to September 2019). In each study, sperm centrioles were assessed near the time of collection after the sperm had been separated into a pellet and an interface using differential gradient centrifugation. In study 1 (November 2016-December 2016), the pellet sperm from two proven fertile donors (D1p and D2p) and 12 patients (P1p-P12p) for a total of 14 consenting men obtained from the Yale Fertility Center was analyzed. In study 2 (May 2018-September 2018), the pellet (P13p-P18p) and interface (P13i-P18i) sperm of 7 infertile men from the Urology Clinic at the Regency Medical Center in Toledo were received and analyzed. In study 3 (May 2019-September 2019), the pellet (P19p-P31p) and interface (P19i-P31i) sperm of 12 infertile men from the Urology Clinic at the Regency Medical Center in Toledo were obtained and analyzed. Centrioles were assessed by confocal microscopy using antibody labeling of POC1B and tubulin.

Semen Samples

Semen samples from consenting and de-identified donors or consenting infertile men were produced by masturbation and ejaculated into containers at home or in the privacy of a clinic room. The ejaculates were allowed to liquefy for at least 30 min at 37° C., and basic semen analysis was performed following WHO guidelines, including information on semen volume, sperm count, motility, and morphology. This information was used to determine which patients had teratospermia or other sperm infertility phenotypes (Table 2). Samples were frozen in sperm cryopreservation media (TYB, Irvine Scientific) following the manufacturer's instructions until use or, were used within five hours of collection (study 2), or were preserved using Sperm CryoProtect (Nidacon, SCP-020) and stored in liquid nitrogen until use (study 3).

TABLE 2
Summary of sperm characteristics from semen analysis.
P values are from T test comparing Infertile with Normal morphology.
SD, standard deviations; N, number; P, p-value; and NA, not available.

| | Infertile | Infertile | Donor |
|---|---|---|---|
| | Normal morphology | Abnormal morphology | Normal morphology |
| | (Range, Mean ± SD, N) | (Range, Mean ± SD, N, P) | (Range, Mean ± SD, N, P) |
| N | 22 | 9 | 2 |
| Age | 19-46, 33.2 ± 6.6, 17 | 27-46, 35.6 ± 7.9, 9, 0.42 | NA |
| Sperm number | 11.7-418, 222 ± 142, 10 | 82-579, 154 ± 233, 8, 0.46 | NA |
| Sperm concentration | 7.8-209, 66.2 ± 61.2, 20 | 0.3-193, 47.7 ± 73.6, 9, 0.47 | 40-228, 134 ± 132.9, 2, 0.19 |
| Progressive motility | 13-58, 35.9 ± 18.1, 10 | 0-36, 15.9 ± 13.1, 8, 0.019 | NA |
| Normal morphology | 5-16, 8.2 ± 3.6, 9 | 0-3, 1.2 ± 1.5, 9, 0.0001 | NA |

Differential Gradient Centrifugation, Washing, Attachment, and Fixation

All samples were separated into interface and pellet sperm using differential gradient centrifugation according to the manufacturer's instructions. The fresh samples were kept at 37° C. (study 2). Frozen samples were thawed at 37° C. (studies 1 and 3). Then, the samples were layered onto a PureCeption gradient (Origio, ART-2040 and ART-2080) (studies 1 and 2) or a 40/80 PureSperm gradient (Nidacon, PS40-100 and PS80-100) (study 3). The interface was separated from the media and discarded (Study 1) or placed in a separate tube (studies 2 and 3) while the pellet remained in the original tube. The interface and pellet sperm were washed using sperm washing media (Origio, ART-1006 (study 2) or Nidacon, PSW-100 (study 3). The final pellet of interface sperm and pellet sperm was resuspended in PBS (study 1) or M199 medium (Sigma-Aldrich, M7528-500ML) (studies 2 and 3). The total volume of resuspended sperm was 100-600 μL based on the size of the pellet. Approximately 30 μL (study 1), 45 μL (study 2), or 35 μL (study 3) of suspended sperm were pipetted into a single well of a Silicone Isolator (EMS 70339-25) on each of 5 Poly Lysine Slides (VWR 16002-116) (study 1), or 6 (Study 2), or at least 3 (study 3), or 8 mm coverslips for each sample (Studies 2 and 3). These slides (study 1) or coverslips (studies 2 and 3) were set to rest for 20 minutes at room temperature, then the excess liquid was removed, and the silicone isolator was removed (Study 1). Either 3.7% formaldehyde (study 2) or methanol (studies 1 and 3) was added as a fixative, and samples were left to rest for 15 minutes at room temperature (study 2) or 5 minutes at −20° C. (studies 1 and 3). Excess fixative was removed. Slides were then washed three times in PBS before mailing in PBS at room temperature to Toledo, where they were stored at 4° C. (study 1) or the coverslips were immediately stained after fixation (studies 2 and 3).

Development of Fluorescence-Based Ratio Assessment of Centrioles (FRAC)

The contribution of the sperm's remodeled centrioles to subfertility and infertility is unknown. To begin resolving this, developing a localization-based, quantitative immunofluorescence assay that is sufficiently robust to compare results from three distinct studies, executed at different times, by different people was desired. Developing a quantitative assay with a continuous spectrum of values for the determination of centriole quality, instead of a categorical/dichotomous determination of centriole defects as present/not present would be useful. A localization-based approach was of interest, as it was found that the two sperm centrioles each have a distinct composition and structure, indicating that different levels of proteins are needed in each centriole for its activity. An immunofluorescence-based assay was of interest as it provides information on the specific components of each of the centrioles separately. Immunofluorescence allows for quantifying multiple centriolar proteins, providing for a more defined analysis of centriole function and contribution to infertility. It can also be used in combination with other fluorescence tools in a multiparametric analysis that tests multiple aspects of sperm function at the same time.

Sperm Staining

Staining was performed at the University of Toledo. Samples were permeabilized with 0.3% Triton X100 in PBS for 1 hour and blocked with 1% BSA in PBS with 0.3% Triton X100 for 2 hours (study 1) or 30 minutes (studies 2 and 3). Primary antibodies diluted in PBS with 1% BSA and 0.3% Triton were applied to the slide (study 1) or coverslip (studies 2 and 3) and incubated overnight at 4° C. Primary antibodies were anti-POC1B (10537, 1:100) and sheep anti-Tubulin (Cytoskeleton, Inc.) (1:600-2,000). Slides were then washed in PBS with 0.3% Triton X-1000 3 times for five minutes each. Subsequently, slides were incubated with secondary antibodies and 1 ug/100 μL Hoechst. The secondary antibodies were donkey anti-rabbit DyLight 650 (Thermo Fisher Scientific, SA5-1004; 1:300-400) or donkey anti-rabbit Alexa 488 (Jackson ImmunoResearch, 711-545-152; 1:300); and donkey anti-Sheep Cy3 (Jackson ImmunoResearch, 713-165-003; 1:1,200-2,000) or donkey anti-Sheep Alexa 555 (Thermo Fisher Scientific, A-21436; 1:1000). Samples were incubated in this solution at room temperature for 4 hours (Study 1) or greater than three hours (Studies 2 and 3), then washed in PBS with 0.3% Triton X100 3 times for 5 minutes each, and PBS 3 times for 5 minutes each. Coverslips were mounted with ProLong gold (Thermo Fischer Scientific, P10144) and allowed to cure for at least 24 hours at room temperature in the dark (Study 1), or were mounted with Vectashield (Vector Laboratories, H-1200) and sealed with clear nail polish (Studies 2 and 3).

Confocal Microscopy

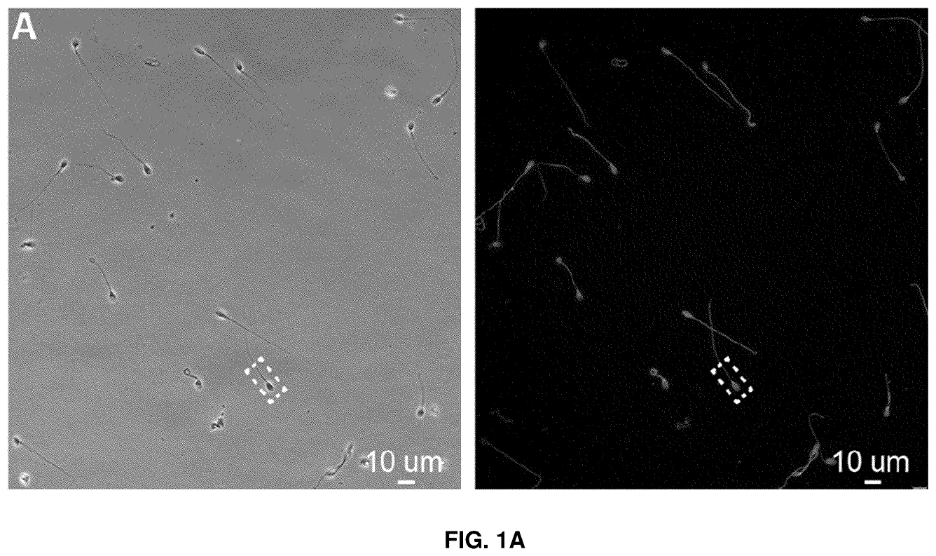

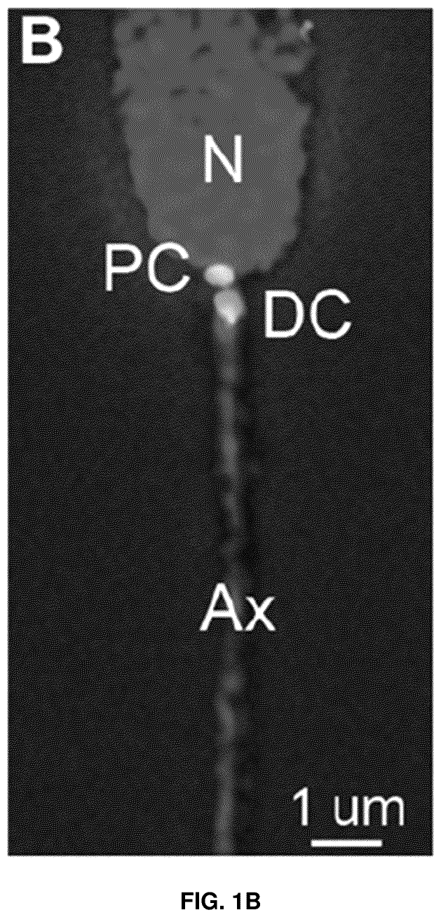

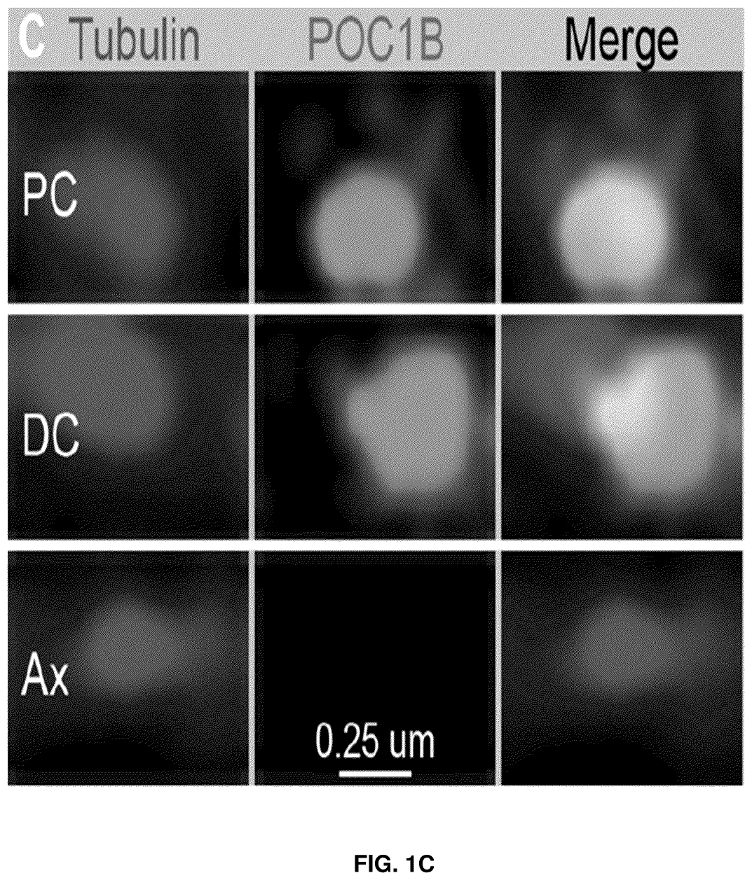

Slides from all three studies were imaged using a Leica SP8 Confocal microscope in photon counting mode using a 630× magnification and a format of 4096×4096 pixels. Some pictures (FIG. 1C) were deconvoluted with HyVolution II from Leica Microsystems.

Staining Quantification

The staining intensities for each marker in the acquired images were quantified in the PC, DC, and Ax of each sperm using a 0.75 um circle (study 1) or a 0.5 um×0.75 um rectangle (studies 2 and 3) (FIG. 1). For every sample, every sperm in the view was measured, regardless of an individual's sperm phenotype (except for one man whose centrioles were unidentifiable).

Calculation of Ratios, Mean Ratios, and the Mean Ratio Range

The mean immunostaining intensity ratio (for simplicity, the “mean ratio”) was determined for each centriolar marker (POC1B and Tubulin), and at each location (PC/total, DC/total, and Ax/total), resulting in 6 mean ratios for each patient (FIG. 1). Since both pellet and interface populations were analyzed, a maximum of 12 mean ratios (6×2=12) per patient can be obtained. Only the pellet fraction was analyzed in study 1. The mean ratios of the 22 men in the reference pellet sperm population were used to generate the reference range. The mean of the mean ratios and the standard deviation (SD) for each marker at each location were calculated; the reference range is the mean±2SD for each marker at each location. The narrowness of the mean ratio range of the mean ratios was determined by subtracting the highest mean ratio from the lowest mean ratio (e.g., for PC tubulin, 0.57-0.25), then dividing that value by the maximal range (i.e., 1), and finally multiplying by 100 to obtain percentage values (i.e., (0.57−0.25/1)*100=32%) (Table 3). The combined mean ratio range was calculated by combining the variation ratio in the PC, DC, and Ax, and then dividing this sum by the number of sites (i.e., for tubulin, (32%+20%+36%)/3)=29%). The more sensitive marker, with the smallest combined mean ratio, was POC1B (13%), while it was more than twice this value for tubulin (29%).

TABLE 3
POC1B is a better marker than tubulin. The table shows the
centriole marker mean ratio range of the reference population
at the PC, DC, and Ax, as well as centriole marker combined
mean ratio range in the pellet sperm (Mean).

| | Tubulin | Tubulin | Tubulin | POC1B | POC1B | POC1B |
|---|---|---|---|---|---|---|
| | Highest | Lowest | Range | Highest | Lowest | Range |
| PC | .57 | .25 | 32% | .44 | .31 | 13% |
| DC | .48 | .28 | 20% | .68 | .49 | 19% |
| Ax | .40 | .04 | 36% | .09 | .01 | 8% |
| Mean | | | 29% | | | 13% |

Statistical Analysis

Normal distribution was determined after calculating the skewness and kurtosis by the functions SKEW and KURT in Excel. Pearson Correlation R and best fit were calculated by the function CORREL and the automatic linear regression in Excel, respectively. The Person Correlation R2 was calculated by RSQ, and the Person Correlation P-value was calculated using the function TDIST in Excel. N was calculated using the function COUNT, the T statistic using the equation (R*SQRT(N−2))/(SQRT(1−R\hat{ }2)), and the degrees of freedom was calculated by N-2, in Excel.

This example used surplus patient material from consenting men undergoing routine medical procedures. Study 1 was approved by Yale's Human investigation committee (HIC 1209010805) and expedited by The University of Toledo's IRB Board (IRB 201599). The University of Toledo's IRB Board approved studies 2 and 3 (IRB 202366).

### Example II—Bovine Sperm Analysis

Most mammalian sperm, like humans and bovines, have two sperm centrioles: the PC with a canonical structure, and the DC, which has an atypical structure and forms the sperm tail's axoneme (Ax). The sperm centrioles are important for fertility, as they participate in sperm motility and embryo development in bovine. However, the contribution of the sperm's remodeled centrioles to subfertility has been unknown. It has been unknown whether centriole assessment can help predict bull subfertility.

A Ratiometric Immunofluorescence Assay for Sperm Centrioles

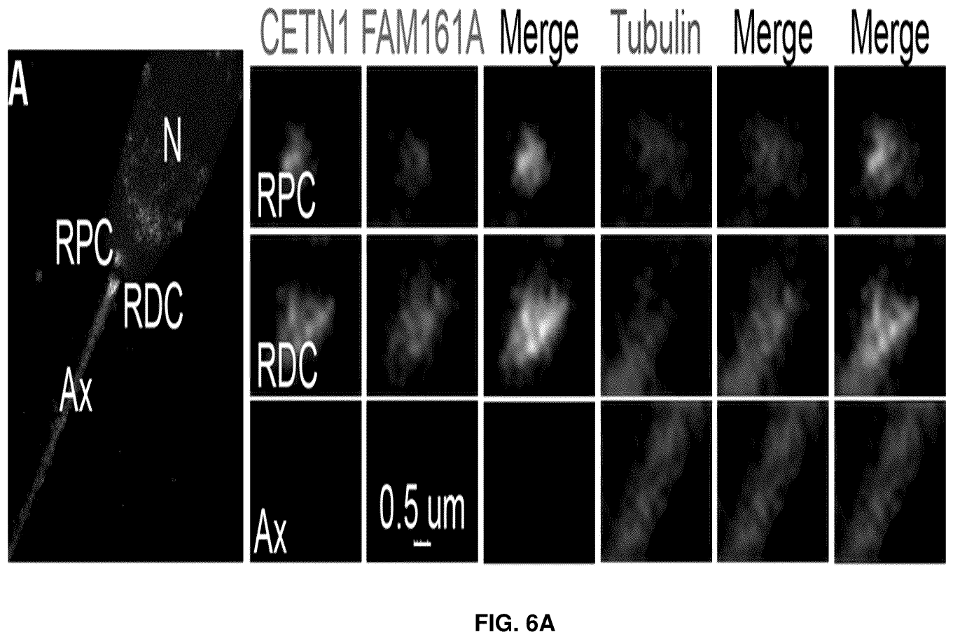

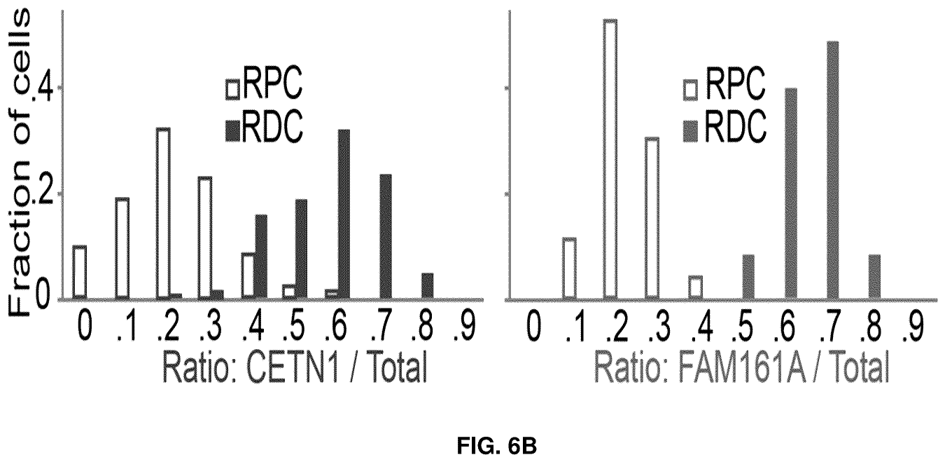

To begin resolving the contribution of sperm's remodeled centrioles to subfertility, a quantitative immunofluorescence-based assay was developed to compare mean staining intensity ratios and, thereby, relative protein levels in the bovine sperm centrioles (FIGS. 6A-6B). The use of these mean ratios is advantageous because it reduces variability due to staining differences between independent staining times, semen samples, and individual sperm.

The quality of sperm centrioles was determined in five steps. First, the staining of each biomarker was quantified in the PC, DC, and Ax in each sperm. Second, the ratios between the staining of PC, DC, or Ax biomarker and the total staining of the PC, DC, and Ax were calculated for each sperm. Third, the mean of each of these ratios per population was calculated. For simplicity, this mean population ratio is named the “mean ratio”. Because three locations in the sperm (PC, DC, and Ax) are looked at, and four biomarkers were analyzed, a maximum of 12 parameters for each sire could be obtained. Fourth, the normal sperm population distribution range was determined by calculating the mean±2SD of the normal sperm (pellet population) of fertile series (having positive SCR). Sperm populations that were within the mean±2SD were considered to have normal centriole staining. Sperm populations that were outside the boundaries of normal distributions were considered outliers and to have abnormal centriole staining Fifth, a sire with normal centriole staining at all 12 parameters in both normal and abnormal sperm populations was considered to have high-quality sperm centrioles. A sire with one abnormal centriole staining at any of the 12 parameters in either normal or abnormal sperm populations was considered to have low-quality sperm centrioles.

Centriole Biomarkers have a Narrow Distribution in Normal Sperm of Fertile Sires

TABLE 4
Centriole biomarker variation at the PC, DC, and Ax, as
well as centriole biomarker mean variation range (Mean)

| | Tubulin | Act Tubulin | POC1B | FAM161A |
|---|---|---|---|---|
| PC | 25% | 9% | 9% | 8% |
| DC | 13% | 20% | 10% | 8% |
| Ax | 12% | 1% | 1% | 0% |
| Mean | 17% | 13% | 7% | 5% |

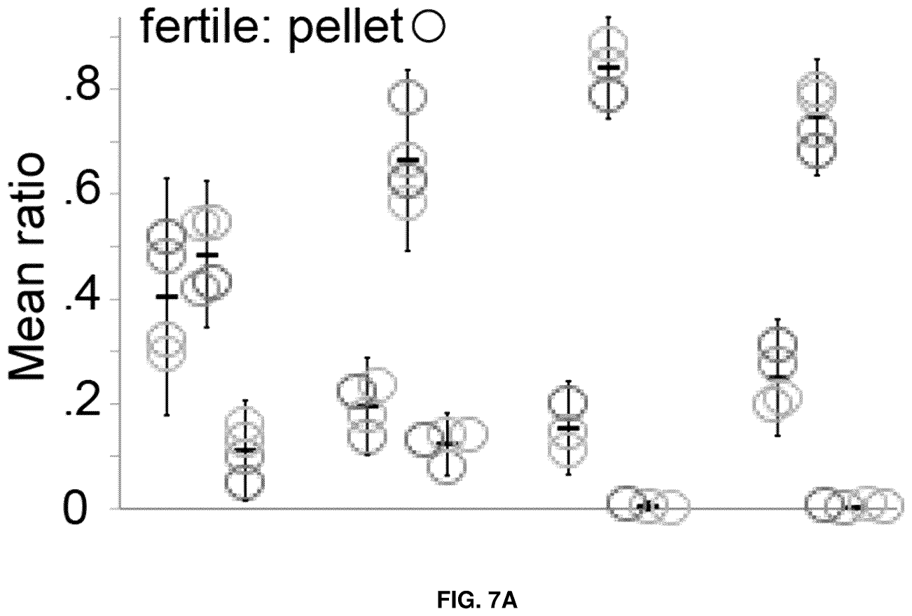

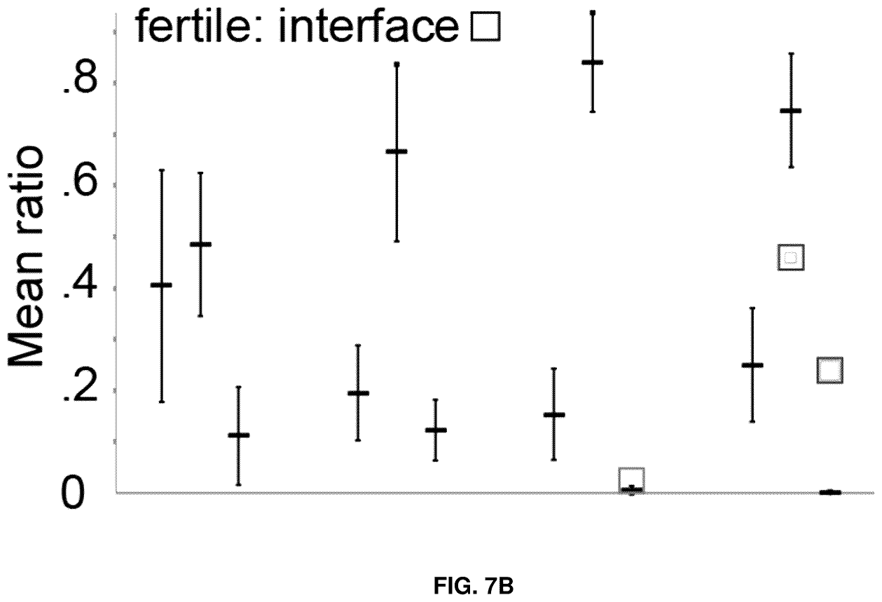

Four fertile Holstein sires that have positive SCR of 2, 2, 2.4, and 2.8 were analyzed. Four sperm centriole biomarkers (tubulin, acetylated tubulin, POC1B, and FAM161A) were used. It was found that the mean ratios of the biomarkers in the normal sperm populations of fertile sires had a narrow distribution range (empty circles in FIG. 7A). To quantify the distribution range of the mean ratios, the highest ratio was substrated from the lowest ratio (e.g., for PC tubulin, 0.55-0.30), then divided that value by the maximal range (i.e., 1), and finally multiplied by 100 to get the values in percentages (i.e., ((0.55−0.30)/1)*100=25%). These calculations indicate that the various ranges were between 0% and 25% in the various conditions and were specific to each biomarker and site combination. (Table 4.)

Next, to identify the most sensitive biomarker (i.e., with the lows over all variation), the biomarker mean variation range was calculated. For that, the variation ratio in the PC, DC, and Ax was combined, and then this sum was divided by the number of sites (i.e., for tubulin, (25%+13%+12%)/3=17%). The most sensitive biomarker, with the smallest mean variation, was FAM161A (5%), followed by POC1B (7%), Act tubulin (13%), and finally, tubulin (17%). These findings indicate that all four biomarkers tested are useful, and the best biomarker is FAM161A.

FIG. 7B shows the mean ratio of abnormal sperm populations of the fertile sires relative to the distribution of the normal sperm populations of the fertile sires.

Low Quality Centrioles Associate with Bull Artificial Insemination Subfertility

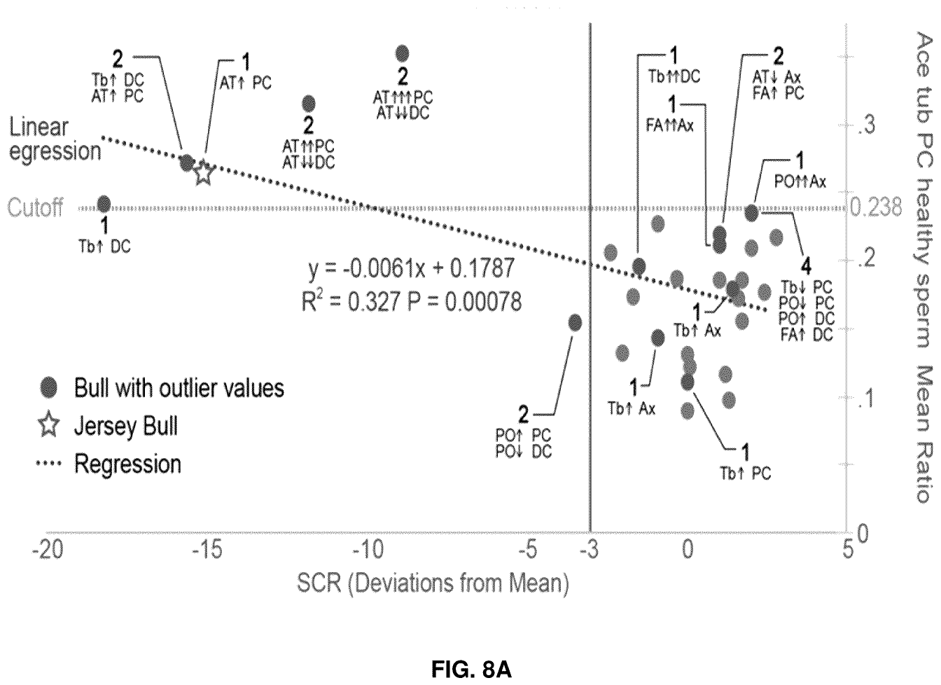

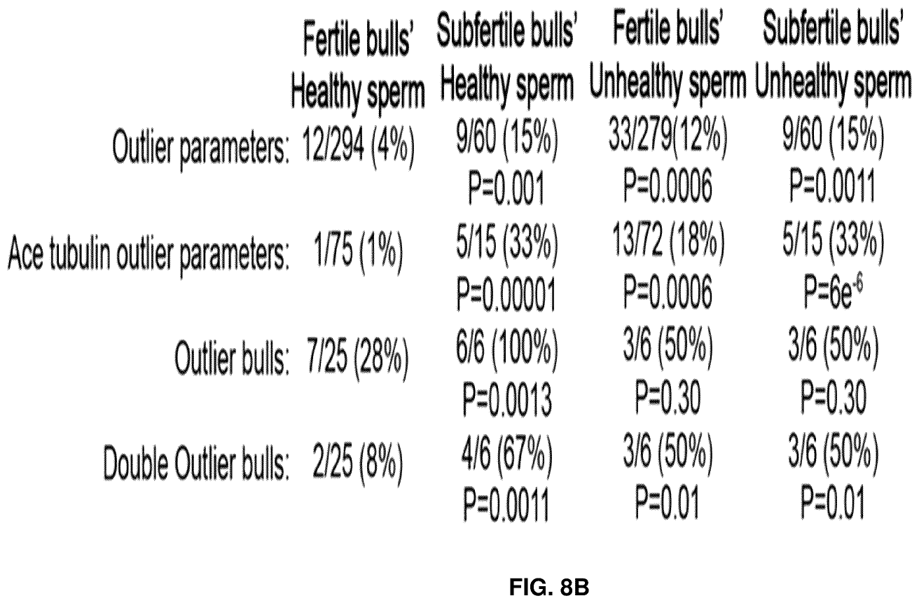

Semen samples from 31 bulls that passed rigorous semen analyses and had a sire conception rate (SCR) ranging between −18 and +3 were analyzed. Of these, 25 were fertile bulls (SCR≥−3), and six were subfertile bulls (SCR<−3). Using differential gradient centrifugation, each semen sample was separated into healthy (pellet) and unhealthy (interface) sperm fractions. Healthy sperm from fertile bulls was used as the reference population. FRAC with four centriole biomarkers (tubulin, acetylated tubulin, POC1B, and FAM161A) was used to determine centriole quality. FRAC found statistically significant lower centriole quality in subfertile bulls' healthy sperm (P=0.001) and unhealthy sperm (P=0.001), and fertile bulls' unhealthy sperm (P=0.0006) when compared to healthy fertile sperm that was used as the reference population (FIG. 8A).

FRAC found similar centriole quality in unhealthy fertile sperm and unhealthy subfertile sperm (P=0.50). The number of outlier parameters in the four sperm populations was counted. It was found that the marker that most commonly contains outliers is acetylated tubulin, indicating it has the best ability to identify centriole quality reduction. Consistently, a significant (P=0.00078) linear regression correlation between SCR and acetylated tubulin in the healthy sperm PC (FIG. 8B) was observed. In the PC acetylated tubulin, the SCR score determined about ⅓ of the variance observed (R2=0.327). Based on this correlation, a 0.238 cutoff of acetylated tubulin PC Pellet Mean Ratio identified five out of the six subfertile bulls and 0 of the 25 fertile bulls. This cutoff value has a sensitivity (the true positive rate) of 83% (5/(6), and the specificity (true negative rate) is 100% (25/25) in the study population. Together, this data indicates that FRAC can help identify bulls with lower sperm quality that is not recognized by current rigorous semen analyses.

Conclusion

The data in this example shows that suboptimal centrioles are common in the subfertile sire, and that bull subfertility is associated with low-quality centrioles. Thus, picking a sire with high-quality centrioles can have a positive impact on sire fertility.

### Example III—Dog Sperm and Horse Sperm

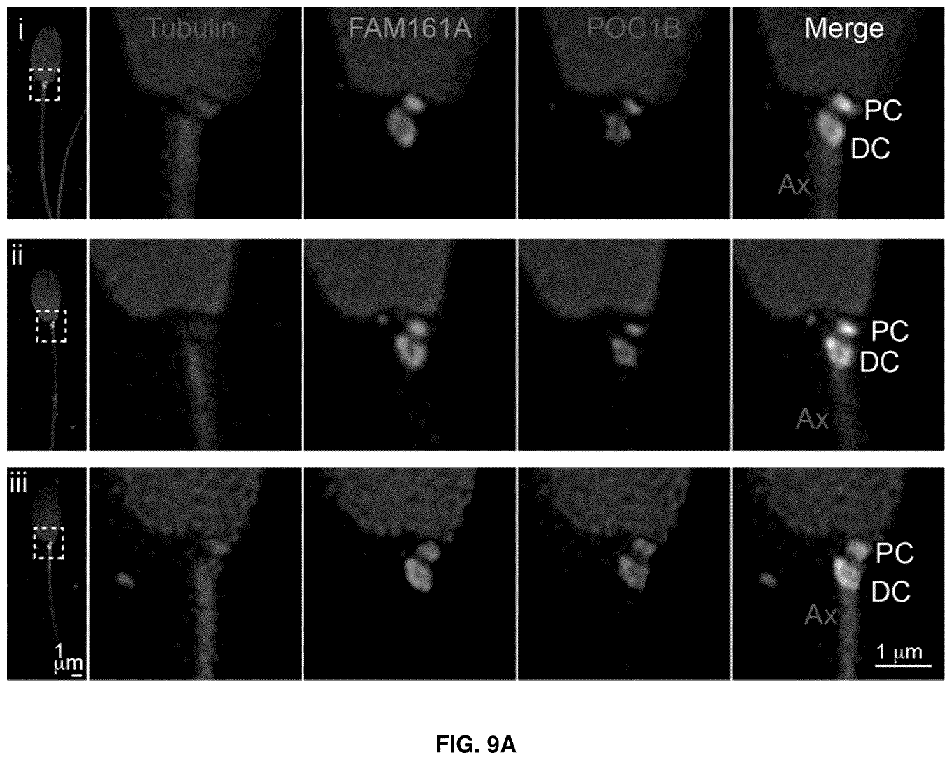

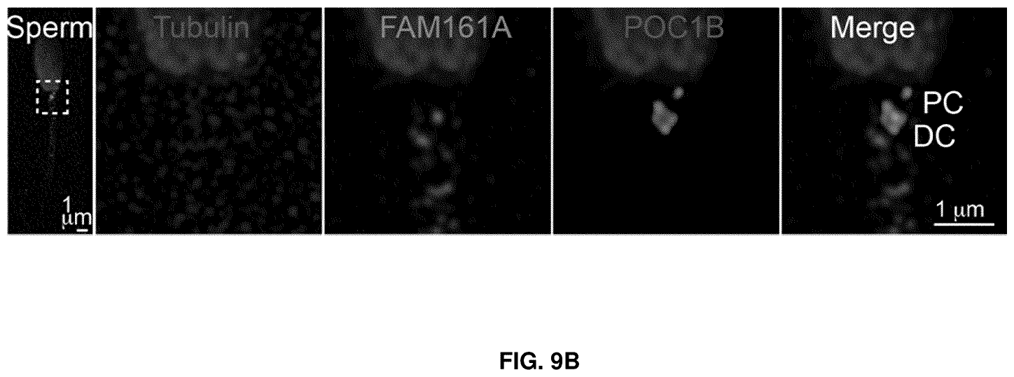

Dog and horse sperm have a proximal centriole (PC) and distal centriole (DC). The DC has two rods that are bigger than the PC lumen. FIGS. 9A-9B show a low magnification of sperm with a doted box marking the magnified neck depicted in the four other panels to the left.

FIG. 9A depicts dog sperm centrioles labeled by the microtubule protein tubulin and the centriole rod/lumen proteins FAM161A and POC1B; tubulin also marks the axoneme of the flagellum. Three sperm cells are shown (i, iii, and iii).

FIG. 9B depicts horse sperm centrioles that are labeled by the centriole rod/lumen proteins POC1B; tubulin and FAM161A do not mark the axoneme and centrioles, because of issues with antibody specificity.

The primary antibodies used in this experiment were rabbit FAM161A (Sigma), mouse POC1B (Thermo fisher), and sheep tubulin. The secondary antibodies used were anti-rabbit Alexa 488, Anti-mouse Alexa 647, Anti sheep Cy3.nti sheep Cy3.

### Example IV—FRAC Assay Robustly and Quantitatively Identifies a Wide Range of Male Factor Infertilities

Unexplained infertility is the failure to identify the cause of infertility in infertile couples and affects about one-third of infertile couples. One of the reasons the causes of infertility are not determined is the lack of robust and sensitive assays for sperm quality that report on the status of the sperm's many essential components. The FRAC assay was developed to determine sperm centriole quality and showed that infertile men with teratospermia have lower quality centrioles in comparison to infertile men with eumorphic sperm.

In this example, it is shown that FRAC can identify lower quality centrioles in men from couples with unexplained infertility. Ten fertile men with healthy sperm selected by centrifugation were the reference population. In this population, the centriolar biomarkers, tubulin, acetylated tubulin, and POC1B had a narrow distribution in the three studied sites (PC, DC, and Ax) and the scores ranged from 8-16% of the FRAC dynamic range.

The FRAC scores of the healthy fertile sperm were approximately normally distributed, and the scores of all ten men were within two standard deviations of the mean. The unhealthy fertile sperm (separated from healthy sperm by density centrifugation) had sub-optimal centrioles; six out of the ten unhealthy fertile samples had suboptimal centrioles (6/10 versus 0/10 in healthy fertile sperm, P=0.003), indicating that unhealthy sperm had lower quality centrioles in fertile men. The healthy sperm from eight of the ten men with unexplained infertility had a sub-optimal centrioles (8/10 versus 0/10, in healthy fertile sperm, P=0.00026), indicating these 8 men had lower quality centrioles and had male factor infertility.

A reduction in centriole quality was also observed in the unhealthy sperm of men with unexplained infertility compared to unhealthy sperm of fertile men by comparing the number of men with parameters that are more than three standard deviations from the mean (5/10 vs. 0/10, P=0.01).

Overall, FRAC identified three levels of centriole quality in the ten unexplained infertility patients: two men had normal sperm centrioles, four men had slightly reduced centriole quality (with one pellet outlier parameter), and four men had mildly reduced centriole quality (with either multiple outlier parameters and mean ratio above three standard deviations in either or both healthy and unhealthy fractions). Finally, a FRAC meta-analysis indicates that FRAC is a robust method to identify various male factor infertility forms.

Infertility, the inability to conceive after 12 months of trying, affects about 10% of couples. About ⅓ of infertile couples have a female factor and another 1/3 have a male factor. In the remaining 1/3 of couples, the cause is unknown, and may be due to a male or female contribution, or a combination of the two. These couples are diagnosed as unexplained couple infertility, or unexplained infertility in short. Unexplained infertility is defined as the absence of identifiable causes for infertility.

Current male fertility examination is based on patient history review, physical and hormonal examinations, and semen analysis. Semen analysis provides general information such as sperm number, motility, and morphology. This analysis only looks at some of the properties of the sperm and, in general, does not strongly correlate with a man's fertility potential. Undetected male factors may be identified by analyzing the sperm's contents that are essential for sperm function such as DNA, RNA, proteins, and structures (e.g., centrioles). Such an analysis is named sperm analysis.

The extent that the various sperm content types are affected in unexplained infertility is unknown. However, centrioles were compromised in some cases of unexplained infertility. Transmission electron microscopy was used and found that a longer sperm centriolar adjunct is associated with unexplained infertility in two affected patients. In this example it is shown that FRAC found reduced centriole quality in 80% of men with unexplained infertility, indicating they had male factor infertility.

Centriolar Biomarker

Tubulin and POC1B are structural components of the centriole and axoneme. Tubulin is a heterodimer complex that polymerizes to form the centriole and axoneme wall. POC1B is an evolutionarily conserved centriolar protein that forms a luminal scaffold structure in canonical centrioles and novel rod structures in the atypical DC and is essential for centriole stability. Acetylated tubulin is a post-translational modification of tubulin. This modification is thought to increase microtubule elasticity and is associated with stable microtubules, such as those found in the centriole and axoneme. The relationship between the microtubules and POC1B rod structures in the DC is unknown, but POC1B likely serves as scaffolding for the distal centriole microtubules. Sperm centrioles are likely acetylated because they are part of a dynamic basal complex that is under large mechanical stress. In this example, three biomarkers were analyzed (Tubulin, POC1B, and acetylated tubulin), and each of them quantifies in three locations (PC, DC, Ax) for a total of nine parameters.

Population Definitions

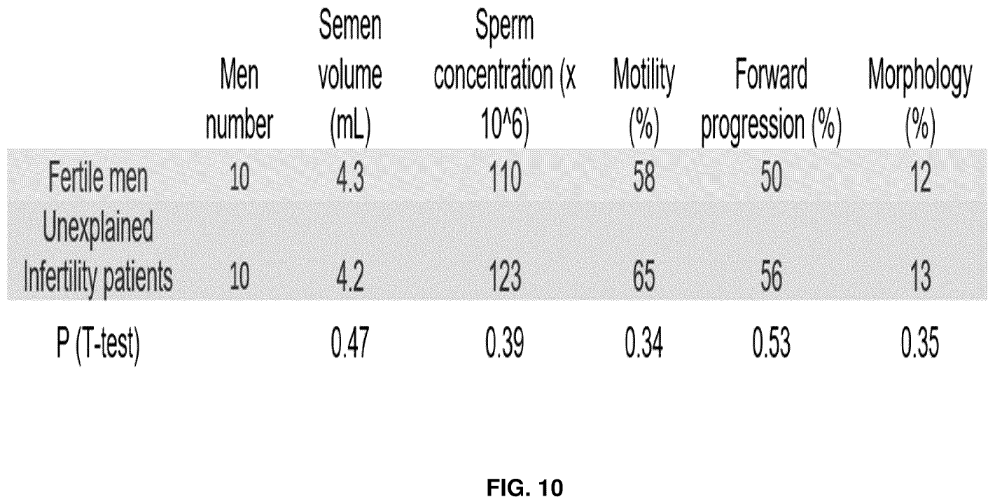

A total of twenty sperm samples were included in the analysis. Ten samples were obtained from men with previously reported pregnancies (referred to as fertile men) and ten samples were obtained from men that were part of a couple diagnosed with unexplained infertility (referred to as unexplained infertility patient). Samples were obtained from the University of Michigan Reproductive Subjects Registry and Sample Repository (RSRSR). Samples were immediately processed per standard protocol following routine semen analysis by a trained andrologist. Sperm were then cryopreserved in liquid nitrogen. The two groups of men had similar average age and semen analysis parameters (FIG. 10).

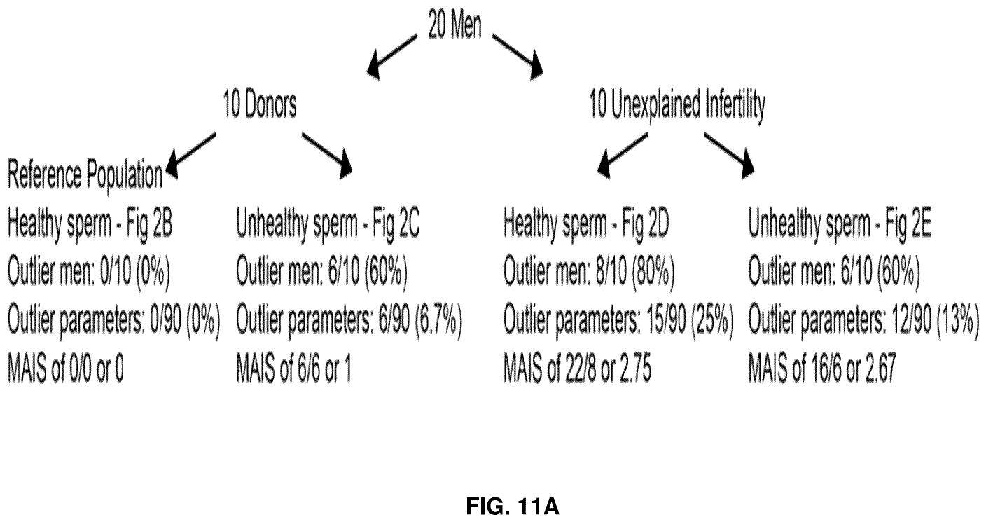

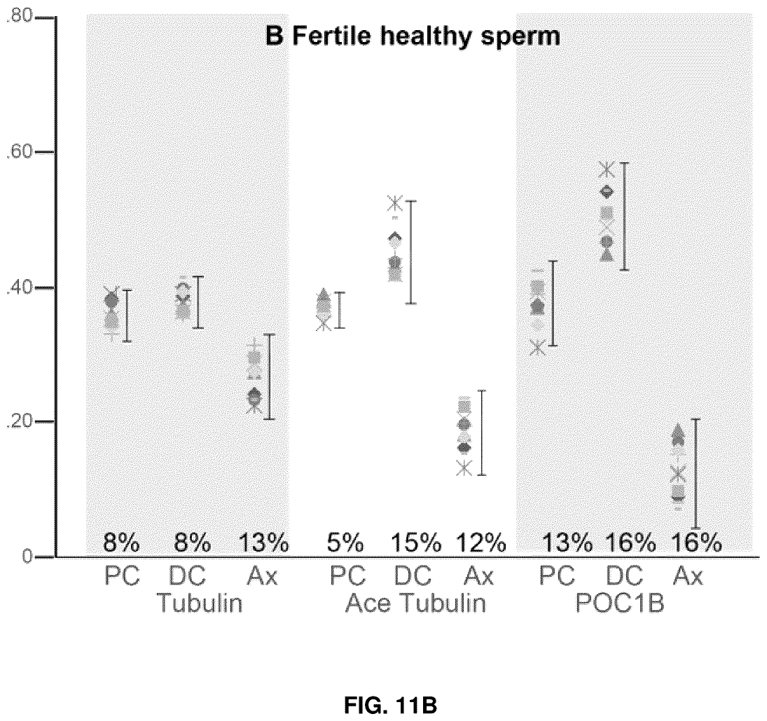

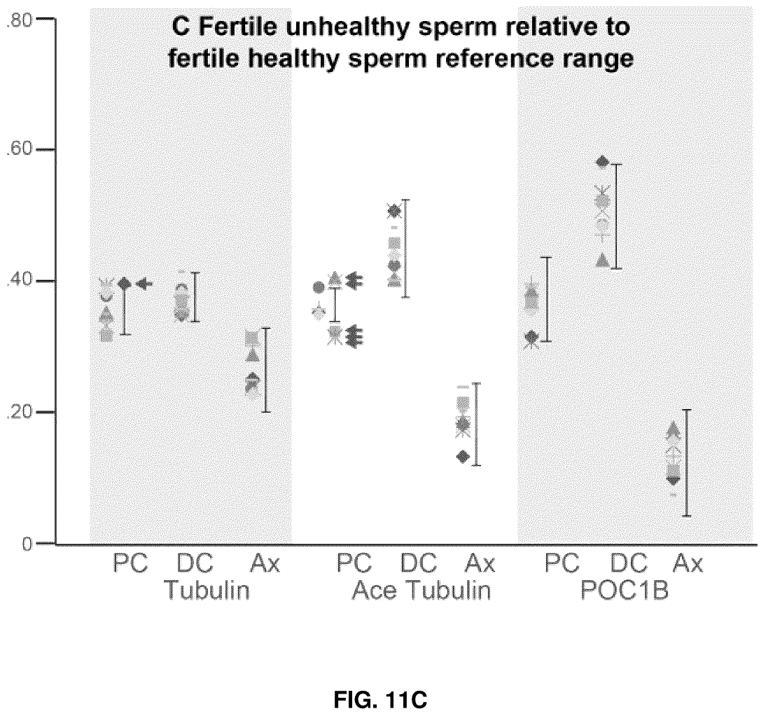

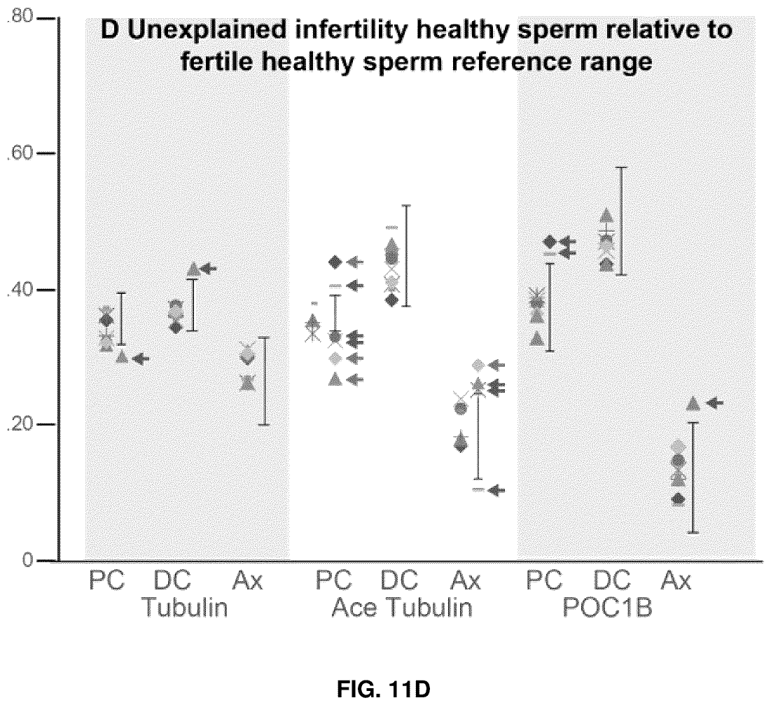

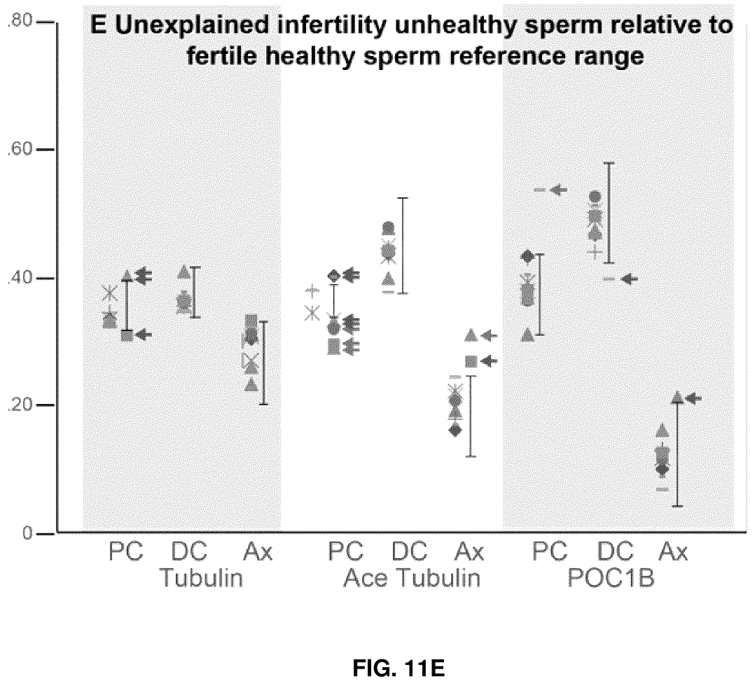

Every cryopreserved sample was processed using gradient centrifugation, which resulted in two fractions: a pellet of dense sperm, generally regarded as higher quality referred to as healthy sperm; and an interface that accumulates lighter sperm having lower quality sperm referred to as unhealthy sperm. This centrifugation produced four groups of sperm, each with hypothetically distinct quality levels (FIG. 11A): i) the fertile healthy sperm (FIG. 11B); ii) the fertile unhealthy sperm (FIG. 11B); iii) the healthy sperm from patients with unexplained infertility (FIG. 11C); and iv) the unhealthy sperm from the patients with unexplained infertility (FIG. 11D). These four groups allowed for the characterization of the differences in sperm centriole quality between men with and without unexplained infertility. The fertile healthy sperm will be referred to as the reference population (FIG. 11A).

FIGS. 11A-11E show that FRAC identifies suboptimal centrioles in unexplained infertility. FIG. 11A shows a breakdown of the 4 populations used. MAIS, mean affected individual score. In FIGS. 11B-11E, for each panel and parameter, the mean and two standard deviations (SD) of the reference population (fertile sperm from pellet fraction) are indicated to the left of the parameter mean ratios within the reference population range. The parameter outliers' values are above and below the reference population, the mean, and two standard deviations (SD). The red arrow indicates values that are above 2SD and below 3SD from the mean. The purple arrow indicates values that are above 3SD from the mean. FIG. 11B shows mean ratios of the fertile pellet sperm (the reference population) and their reference range. FIG. 11C shows mean ratios of the fertile interface sperm relative to the reference range. FIG. 11D shows mean ratios of the pellet sperm with unexplained infertility relative to the reference range. FIG. 11E shows mean ratios of the interface sperm with unexplained infertility relative to the reference range.

Fertile Pellet Sperm FRAC Score had Approximately Normal Distribution

The reference population was made of fertile men with prior paternity with a normal semen analysis. The variation of the mean ratios within the reference population was analyzed to determine the range of good quality centrioles. The distribution of the mean ratios in the reference population was normal. Normality was tested for, and it was found that the five tests used did not reject the null hypothesis. Also, the skewness was normal (between −1 and +1), and the kurtosis was normal (between −3 and +3).

The standard deviation (SD) of the nine parameters ranged between 0.019 and 0.040 and overall was very low (below 3% of the assay dynamic range of 0 to 1). The mean SD was lowest for tubulin (the mean of the 3 locations is 0.023±0.007), intermediate for acetylated tubulin (0.027±0.013), and highest for POC1B (0.037±0.005). The differences between the protein mean SDs are not significant, but the difference between the tubulin and acetylated tubulin mean SDs is almost significant (P=0.053).

To identify the samples with a 95% probability of belonging to the reference population, the mean of the ratios in the reference population±two standard deviations (SDs) was calculated, which is referred to as the reference range (FIG. 11B).

The FRAC method has a minimal score of 0 and maximal score of 1; the range between this minimal and maximal score defines the FRAC dynamic range. The reference population for the nine parameters ranged from 5%-16% with POC1B having ranges of 13%, 16%, and 16%, tubulin having ranges of 7%, 8%, and 13%, and acetylated tubulin having ranges of 5%, 15%, and 13% of the assay dynamic range. The range of the fertile donor is a bit narrower than that of infertile men with eumorphic sperm (POC1B: 14, 21, 20%; Tubulin: 8, 9, and 12%; P=0.05).

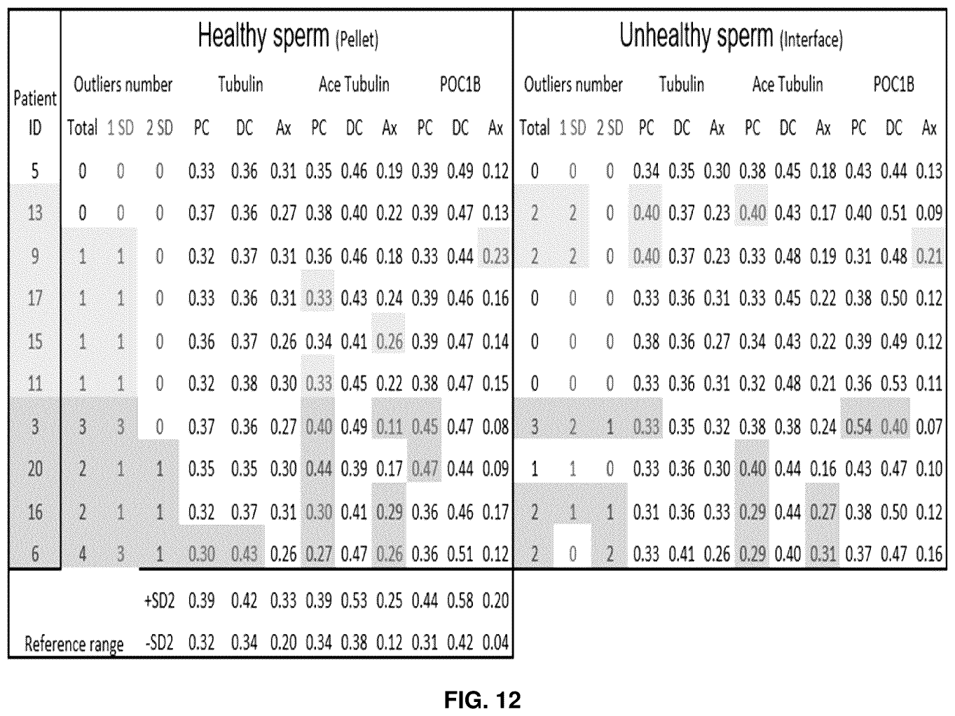

Samples with a mean ratio outside the reference range for any of the nine mean ratio locations/markers were considered outliers and deemed to have sub-optimal centrioles. A sample from the reference population was considered to have optimal centrioles if all nine sperm mean ratios were within the reference range. All ten samples from the reference population had all nine mean ratios fall within the reference range and can be considered to have optimal centrioles (FIG. 12).

Fertile Interface Sperm had Sub-Optimal Centrioles

As sperm cells differentiate, they lose cytoplasm and become denser. This difference is used to select the better sperm found in the pellet in differential centrifugation. During sperm differentiation, centrioles are also being remodeled. Therefore, the less dense and thus less mature sperm found in the interface have reduced centriole quality. Previously, an insignificant increase in the incidence of suboptimal centrioles (4/9 versus 3/22, P=0.06) was found with eumorphic sperm from infertile men. However, in the present example, it was found that six out of the ten interphase samples had suboptimal centrioles (6/10 versus 0/10, P=0.003), indicating that the interphase has lower quality centrioles in fertile sperm.

All of the six samples had a single outlier value that was more than 2 SDs, but less than 3 SDs from the mean. To quantify the severity of the low-quality centriole subpopulations, the Mean Affected Individual Score was calculated. (The numerator is the number of outlier values added together where >2SD is one, and >3SD is two, and the denominator is the number of affected individuals.) It was found that the Mean Affected Individual Score of the low-quality centriole subpopulations of fertile men was 1 (6/6=1).

80% of Men with Unexplained Infertility had Sub-Optimal Centrioles

Some reports indicate that patients with unexplained infertility have defective sperm centrioles. FRAC can identify some men with unexplained infertility having suboptimal sperm centrioles. As such, the objective of this analysis was to test if POC1B, tubulin, and acetylated tubulin can identify centriolar anomalies in unexplained infertility samples.

The mean ratios of the pellet sperm from 10 patients with unexplained infertility were analyzed. It was found that eight men had outlier mean ratios of centriole markers (FIG. 11D). These results indicate that the patients with unexplained infertility have more sub-optimal centrioles than fertile men (8/10, versus 0/10, P=0.00026). Four patients (9, 11, 15, and 17) had a single outlier (2-4 parameter). Four patients (3, 6, 16, and 20) had multiple outliers (2-4 parameters). Three of them (6, 16, and 20) had mean ratios above three SDs from the mean of the reference population for one parameter. These four samples as samples were designated with “severe centriole defects.” There is an almost statistically significant difference in the number of severe centriolar defects in unexplained infertility patients versus fertile men (3/10 versus 0/10, P=0.060).

The Mean Affected Individual Score of healthy unexplained infertility sperm samples (20/8=2.5) was higher than that of healthy fertile sperm (0), and unhealthy fertile sperm (1). This observation indicates that healthy unexplained infertility sperm had lower quality than unhealthy fertile sperm.

Overall, these findings show that sperm from patients with unexplained infertility are more likely to have lower-quality centrioles. These findings also show that FRAC can identify male factor infertility men diagnosed currently with unexplained couple infertility.

Differential Centrifugation is Insufficient to Select Better Centriole Quality in Unexplained Infertility Men

It was found that differential gradient centrifugation of sperm from infertile men with eumorphic sperm results in a healthy sperm enriched for better quality sperm centrioles. The same for fertile sperm was also found (see above). Whether differential gradient centrifugation would have a similar effect on sperm from patients with unexplained infertility was evaluated. Interface sperm from men with unexplained infertility were analyzed (FIG. 11E). It was found that 6 of the 10 interface sperm samples from men with unexplained infertility had mean ratios outside the reference range. This is a similar rate to that in the pellet sperm (6/10 versus 8/10, p=0.3291). Also, the Mean Affected Individual Score was similar in the interface versus the pellet of unexplained infertility samples (16/6=2.67 vs. 22/8=2.75, P=0.32708). This result indicates that infertile patients with unexplained infertility have a similar incidence of sub-optimal centrioles in the interface and the pellet fractions. Furthermore, this observation indicates that sperm selection by differential centrifugation is insufficient for increasing the odds of fertilization in cases of sub-optimal centrioles.

50% of Unexplained Infertility Patients had Interface Centrioles of Lower Quality than Fertile Interface

Unexplained infertility patients' healthy sperm has lower centriole quality than the fertile men's healthy sperm. Therefore, whether the reduced centriole quality is also detected when comparing the unhealthy unexplained infertility sperm was evaluated. It was found that the number of unhealthy fertile sperm with outliers (6/10) is like that of unhealthy unexplained infertility sperm (6/10). However, the Mean Affected Individual Score of unhealthy fertile sperm (6/6=1) is much smaller than that of unhealthy unexplained infertility sperm (16/6=2.66), indicating that the severity of centriole quality reduction is increased in unexplained infertility patient's unhealthy sperm. Indeed, it was found that none of the fertile men's unhealthy sperm had outliers more than 3 SDs away while three unexplained infertility patients' unhealthy sperm had 3 SD outliers (P=0.06). Also, none of the unhealthy fertile sperm had 2 outlier values while 5 unexplained infertility patient's unhealthy sperm had two or more outliers (P=0.01). This observation indicates that 50% of unexplained infertility patients had unhealthy sperm centrioles of lower quality than unhealthy fertile sperm.

FRAC Identifies Three Levels of Centriole Quality in Unexplained Infertility Men

The above finding with the FRAC method indicates that the unexplained infertility patients analyzed fall into one of three categories (FIG. 12). The first category is normal, which is patients with 0 healthy sperm outliers and one unhealthy sperm outlier that is ≤1 SD from the reference distribution. There was one unexplained infertility patient that had normal sperm centrioles in the cohort—patient 5. The second category is possible, which is patients with one healthy sperm outlier that is <1 SD away from the reference distribution; or with 2 unhealthy sperm outliers <1 standard deviation from the reference distribution. Five patients had this slightly reduced centriole quality (9, 11, 13, 15, and 17). The third category is likely, which is patients with two or more healthy sperm outliers that are <1 standard deviation from the reference distribution; or with three or more unhealthy sperm outliers that are <1 standard deviation from the reference distribution; or any number of patients with healthy sperm or unhealthy sperm outliers that are ≥1 standard deviation from the reference distribution. Four patients had this mildly reduced centriole quality (patients 3, 6, 16, and 20).

FIG. 12 represents a table of mean ratios of healthy sperm and unhealthy sperm from unexplained infertility patients. Blue font in FIG. 12 indicates a mean ratio that is <1 SD over reference range. Red font in FIG. 12 indicates a mean ratio that is >1 SD over reference range. Yellow highlights mark the “possible” level. Orange highlights mark the “likely” level.

Meta-Analysis Indicates FRAC is a Robust Method to Identify Male Factor Infertility

To better understand the FRAC capability to identify male factor infertility, the results from the current example were combined with previously obtained data to generate a combined meta-analysis only using Tubulin and POC1B, so in the meta-analysis, the information obtained only by these two biomarkers were compared. This results in 6 parameters.

The study included three types of men (n=33): (I) fertile men (n=2); (ii) infertile patients with eumorphic sperm (normal morphology) (n=22); and (iii) teratospermia (poor morphology) infertile patients (n=9). The current study included 2 types of men (n=20): (i) fertile men (n=10); (ii) infertile patients with unexplained infertility (n=10). Together, the meta-analysis generated four groups of sperm: (i) fertile men (12); (ii) infertile patients with unexplained infertility (n=10); (iii) infertile patients with eumorphic sperm (normal morphology) (n=22); and (iv) teratospermia (poor morphology) infertile patients (n=9).

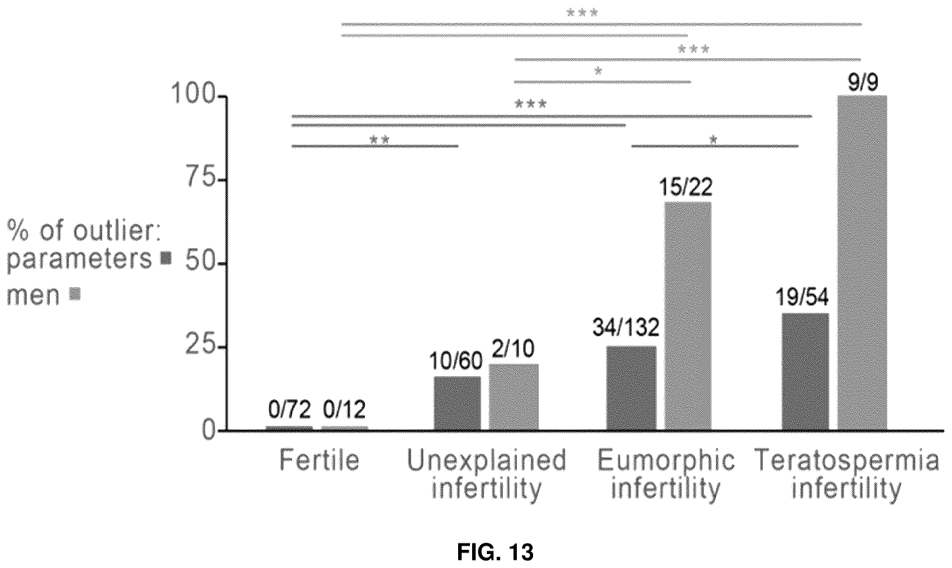

First, the meta-reference population was calculated by combining the mean ratio values of this example's 10 fertile men and the 2 fertile men of the previous study. This new reference population was then compared to unexplained infertility, and infertility patients with eumorphic sperm or teratospermia. Using the combined meta-reference population, it was found that the fertile men pellet had 0 outlier men (0/12, 0%) and 0 outlier values (0/72, 0%). Unexplained infertile patients had 10 outliers' values (10/60=17%) in 2 men (men 6 and 20, 2/10, 20%). Eumorphic infertile patients had 34 outliers' values (34/132=26%) in 15 men (15/22=68%). Teratospermia infertile patients had 33 outliers' values (19/54=37%) in 9 out of 9 men (9/9=100%). Together, this meta-analysis demonstrates that FRAC is a robust test for male factor infertility (FIG. 13).

Discussion

Identifying the male contribution to infertility is critical for improving treatment and women's health. A majority of physicians surveyed asserted that the development of a sperm test to unexplained couple infertility to characterize male-factor infertility would both be helpful clinically and change practice patterns. Also, one of the consequences of failing to recognize the male contribution to infertility is reduced male fertility research and inadequate treatment of male fertility. Therefore, male infertility treatment is currently mainly reached by using assisted reproductive technologies, which are performed in the women's body. An additional consequence is that women appear as the cause of infertility, inducing psycho-social stress in front of their family and communities.

The recent follow-up study of NIH-supported Fast Track and Standard Treatment Trial (FASTT) involving 286 patients indicates that 64% of couples with unexplained infertility were able to have at least one further child without additional medical treatment. With treatment 23% achieved at least one live birth, and 19% of the patients were not able to conceive by the end of the follow-up. Therefore, it is important to determine if advanced sperm analysis can help stratify patients to only those that do not need further treatment, those that are likely to need treatment to conceive, and those that are unlikely to conceive even with currently available treatment.

This example shows a finding of reduced centriole quality in infertile men. This reduced centriole quality in these men is either contributing to infertility or a consequence of the underlying infertility cause. These two possibilities can be distinguished with a more detailed sperm analysis including other sperm factors. Also, centrosome functional tests may be utilized to determine the centriole quality associated with clinical deficiency.

Certain embodiments of the systems, assays, methods, and kits disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the systems, assays, methods, and kits described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

1. A method for diagnosing a likelihood of infertility in a male subject, the method comprising:
staining a sperm sample from a male subject with at least one antibody configured to bind to a centriolar marker at locations comprising each of a proximal centriolar, atypical distal centriole, and axoneme of the sperm, wherein the antibody is configured to fluoresce upon binding to the centriolar marker;
observing fluorescence from the stained sperm sample at each of the locations;
comparing the observed fluorescence at each of the locations to a total fluorescence from the stained sperm sample at all of the locations to obtain a ratio at each of the locations for the centriolar marker;
averaging the ratios by a number of samples to obtain a mean ratio at each of the locations;
comparing the mean ratios to reference mean ratios from a reference sperm sample, wherein the reference sperm sample has standard morphology; and
diagnosing a likelihood of infertility in the male subject when any one of the mean ratios falls outside of two standard deviations from the reference mean ratio at the same location.

2. The method of claim 1, wherein the centriolar marker comprises tubulin.

3. The method of claim 1, wherein the centriolar marker comprises POC1B.

4. The method of claim 1, wherein the centriolar marker comprises tubulin and POC1B.

5. The method of claim 1, wherein the centriolar marker comprises any of tubulin, acetylated tubulin, POC1B, POC5, CPAP, CEP63, CEP290, CETN1, CETN2, FAM161A, WDR90, CEP57, or CEP152.

6. The method of claim 1, wherein the centriolar marker comprises two or more markers selected from the group consisting of tubulin, acetylated tubulin, POC1B, POC5, CPAP, CEP63, CEP290, CETN1, CETN2, FAM161A, WDR90, CEP57, and CEP152.

7. The method of claim 1, wherein the centriolar marker comprises three or more markers selected from the group consisting of tubulin, acetylated tubulin, POC1B, POC5, CPAP, CEP63, CEP290, CETN1, CETN2, FAM161A, WDR90, CEP57, and CEP152.

8. The method of claim 1, wherein the reference sperm sample comprises sperm from an infertile subject with standard morphology.

9. The method of claim 1, wherein the antibody is labeled with a fluorescent tag.

10. The method of claim 1, wherein a secondary antibody is used to configure the antibody to fluoresce upon binding to the centriolar marker.

11. The method of claim 1, wherein the male subject is a human.

12. The method of claim 1, wherein the male subject is a male ungulate species.

13. The method of claim 1, wherein the male subject is a male ruminant species.

14. The method of claim 1, wherein the male subject is a dog, horse, bull, ram, or boar.

15. The method of claim 1, wherein the male subject is a male mammalian food animal species.

16. The method of claim 1, wherein the male subject is a domesticated animal.

17. A system comprising:
one or more antibodies configured to fluoresce upon binding to a centriolar marker selected from the group consisting of tubulin, acetylated tubulin, POC1B, POC5, CPAP, CEP63, CEP290, CETN1, CETN2, FAM161A, WDR90, CEP57, and CEP152; and
a computer system configured to read fluorescence from a stained sperm sample, and process the fluorescence through an algorithm to calculate mean ratios of fluorescence at multiple locations within each sperm.

18. The system of claim 17, further comprising a microscope.

19-21. (canceled)

22. An assay comprising:
one or more primary antibodies configured to bind to a centriolar marker selected from the group consisting of tubulin, acetylated tubulin, POC1B, POC5, CPAP, CEP63, CEP290, CETN1, CETN2, FAM161A, WDR90, CEP57, and CEP152; and
one or more secondary antibodies configured to fluoresce upon binding to the one or more primary antibodies.

23-24. (canceled) (Currently Amended) The method of claim 22, wherein the secondary antibodies comprise donkey anti-rabbit DyLight 650, donkey anti-rabbit Alexa 488, donkey anti-Sheep Cy3, or donkey anti-Sheep Alexa 555.
